(19) United States
(12) Patent Application Publication
(10) Pub. No.: US 2011/0123484 A1
(43) Pub. Date: May 26, 2011

(54) METHODS AND COMPOSITIONS FOR TREATING FUS1 RELATED DISORDERS

(75) Inventors: Michael Isacc Lerman, Rockville, MD (US); Alla Vladimir Ivanova, Frederick, MD (US); Sergey Vladimir Ivanov, Frederick, TN (US)

(73) Assignee: GOVERNMENT OF THE US, AS REPRESENTED BY THE SECRETARY, DEPARTMENT OF HEALTH AND HUMAN RESOURCE, ROCKVILLE, MD (US)

(21) Appl. No.: 11/988,427

(22) PCT Filed: Jul. 7, 2006
(86) PCT No.: PCT/US2006/026533

§ 371 Date: Dec. 15, 2009

Related U.S. Application Data
(60) Provisional application No. 60/697,596, filed on Jul. 7, 2005.

(51) Int. Cl. A61K 38/20; C12N 5/078; C12N 5/0781; C12N 5/0783; C12Q 1/68; G01N 33/68; A61P 7/06; A61P 29/00; A61P 35/00; A61P 37/06

(52) U.S. Cl. 424/85.2; 435/375; 424/184.1; 435/6.1; 436/501

(57) ABSTRACT

The invention relates to methods, systems, and transgenic animals useful for screening, diagnosing, and treating Fus1 related disorders. Further disclosed herein are novel methods for inhibiting cellular proliferation disorders as well as immune system disorders.

### CROSS-REFERENCE TO RELATED APPLICATION

This application claims the benefit of the following U.S. Provisional Application No. 60/697,596, which was filed on Jul. 7, 2005, the contents of which are incorporated herein by reference.

### STATEMENT REGARDING FEDERALLY SPONSORED RESEARCH OR DEVELOPMENT

Research supporting this application was carried out by the United States of America as represented by the Secretary, Department of Health and Human Services.

### BACKGROUND OF THE INVENTION

Fus1 was first characterized by positional cloning in an effort to define the minimal chromosomal region associated with lung cancer. The ˜120 kb region of the human 3p21.3 chromosome that harbors Fus1 and other tumor suppressor gene (TSG) candidates showed allele loss in pre-neoplastic lesions and even in histologically normal bronchial epithelium of current and former smokers. Thus, Fus1 and other TSG candidates that reside in this region may contribute to the earliest steps of lung cancer pathogenesis. Association of the instability in this region with breast, head and neck, and other cancers further substantiates its tumor suppressor properties (1). While analysis of lung cancer cell lines revealed a limited (˜4%) frequency of mutations in the Fus1 gene (1), functional studies proved its growth suppressor properties in vitro (2) and in vivo (3). Furthermore, intra-tumoral administration of Fus1 suppressed experimental lung metastasis in mice, and lung tumor-bearing animals treated with Fus1 showed prolonged survival (3). While it can be inferred from these experiments that the Fus1 product might suppress tumor growth and even possess therapeutic potential, the molecular mechanism(s) of Fus1 action and its biological function remain unknown. The human Fus1 gene encodes a short (110 amino acids) evolutionary conserved protein (93% similarity between mouse and human) that shows no apparent similarity to other proteins. Fus1 is also known as TUSC1 and the mouse Tusc1.

In recent years, there has been a paradigm shift in how cancer is viewed. Researchers have begun to consider the similarities between seemingly divergent malignancies, rather than focusing on the differences between them. Hanahan and Weinberg have summarized this view by proposing that cancer cells must acquire six characteristics in order to form progressively growing tumors (4). Specifically, tumors must be able to grow autonomously, develop insensitivity to negative growth regulation, evade intrinsic apoptotic signals, display unlimited replicative potential, overcome hypoxia through induction of angiogenesis, and attain proficiency for invasive growth and metastasis. In recent reviews, Dunn, Old and Schreiber (5, 6) extend this list by adding the seventh “hallmark of cancer”: the capacity of the malignant cells to evade the extrinsic tumor suppressor functions of the immune system. Recent data obtained by many independent groups overwhelmingly support the basic tenets of the cancer immuno-surveillance concept, namely, that the un-manipulated immune system is capable of recognizing and eliminating primary tumors and that lymphocytes and the cytokines they produce are important in this process.

Natural killer (NK) cells represent a lymphocyte subset that plays a role in innate immunity by mediating two major functions: the recognition and lysis of tumor and virus-infected cells without sensitization and the production of immunoregulatory cytokines, following activation (7). Therefore, genetic, immunochemical, or functional alterations of this subset lead to increased susceptibility of the host to tumors. Thus, mice depleted of NK cells following anti-asialo-GM1 treatment were 2 to 3 times more prone to develop carcinogen-induced tumors than control counterparts (8). Development of NK cells depends on the presence of cytokines using the common receptor gamma chain (γ_{c}), including IL-2, IL-4, IL-7, IL-9, IL-15, and IL-21 (9). However, the dominant γ_{c} cytokine for NK cell maturation is IL-15, since mice deficient in IL-15 or IL-15 receptor lack mature NK cells (10, 11).

### BRIEF SUMMARY OF THE INVENTION

The tumor suppressor properties of Fus1 have been confirmed experimentally by intra-tumoral administration of FUS1, which suppressed experimental lung metastasis in mice. Described herein are Fus1-deficient mice that are viable, fertile, and demonstrate a complex immunological phenotype. Animals with a disrupted Fus1 gene developed signs of autoimmune disease, such as vasculitis, glomerulonephritis, anemia, circulating autoantibodies, and showed an increased frequency of spontaneous vascular and hematopoietic tumors. Fus1 null mice demonstrated a consistent defect in NK cell maturation that correlated with changes in the expression of the IL-15. Injection of IL-15 into Fus1 knockout mice completely rescued the NK cell maturation defect.

Described herein, Fus1-deficient mice were generated and their complex immunological phenotype was characterized. Without wishing to be bound by any particular theory, the Fus1^{-/-} mice present a defect in NK cell maturation caused, either directly or indirectly, by insufficient production of IL-15. Fus1^{+/-} and Fus1^{-/-} animals developed signs of autoimmune disease, such as inflammatory infiltration of vessels, glomerulonephritis (GN), anemia, and showed an increased frequency of spontaneous vascular and hematopoietic tumors.

Provided herein, according to one aspect are methods for inhibiting cellular proliferation comprising contacting an immune cell with a Fus1 polypeptide. Examples of Fus1 polypeptides include, for example, SEQ ID NOs.: 14-15 and polypeptides encoded by SEQ. ID. NOs.: 1-13.

In one embodiment, the Fus1 polypeptide induces apoptotic cell death of the immune cell.

In another embodiment, the immune system cell comprises one or more of an NK cell, T cell, B cell, activated T cell, and activated B cell.

According to one embodiment, the methods may further comprise providing a Fus1 polypeptide. In a related embodiment, the Fus1 polypeptide is obtained from cultured cells. In another related embodiment, the cultured cells comprise an expression construct comprising a nucleic acid segment encoding FUS1. In a further related embodiment, the FUS1 nucleic acid segment is under the control of a promoter. In yet another related embodiment, the polypeptide is produced from an expression construct comprising a nucleic acid segment encoding FUS1 under the control of a promoter.

In one embodiment, the expression construct is a viral or non-viral expression construct. In a related embodiment, the viral expression construct is adenovirus, retrovirus, adeno-associated virus, herpesvirus, vaccinia virus or polyoma virus.

According to one embodiment, the methods further comprise treating the cell with one or more additional anti-proliferative treatments.

Provided herein, according to another aspect, are methods of treating, preventing or alleviating a FUS1 immune related disorder in a subject, comprising administering a FUS1 composition to the subject.

According to one embodiment, the FUS1 related disorder is one or more of an autoimmune disease, anemia, hematopoietic tumor, a virus associated malignancy, inflammatory infiltrating of vessels, glomerulonephritis, vascular tumor, circulating antibodies, vasculitis, lymphoma, or NK cell maturation defect.

In one embodiment, the composition is administered, for example, systemically, intratumorally, intravascularally, to a resected tumor bed, orally, or by inhalation. In a related embodiment, the composition is administered in a single dose. In another related embodiment, the composition is administered in multiple doses. In a further related embodiment, the composition is continuously infused over a period of time exceeding one hour.

According to one embodiment, the methods may further comprise administering one or more additional therapies to the subject. In a related embodiment, the therapy is surgery, chemotherapy, radiotherapy, gene therapy, immune therapy or hormonal therapy.

In one embodiment, an immune cell of the subject expresses a reduced level of FUS1 polypeptide, or fragments or variants thereof.

In another embodiment, the FUS1 composition comprises a substantially purified FUS1 polypeptide, or fragment or variant thereof. In a related embodiment, the FUS1 composition comprises an nucleic acid encoding a FUS1 polypeptide, or fragment or variant thereof. In another related embodiment, the FUS1 polypeptide is under the control of a promoter. In a further related embodiment, the FUS1 composition is a FUS1 expression construct. In yet another related embodiment, the expression construct is a viral or non-viral expression construct. In another related embodiment, the viral expression construct is selected from one or more of adenovirus, retrovirus, adeno-associated virus, herpesvirus, vaccinia virus or polyoma virus. In another embodiment, the non-viral expression construct is encapsulated in a liposome. In yet another embodiment, the expression construct further comprises a promoter. In certain embodiments, the subject is a mammal.

Also provided herein, according to one aspect, are methods for predicting or diagnosing a FUS1 immune related disorder in a subject comprising determining a level of FUS1 expression in a sample from a subject.

Also provided herein, according to one aspect, are methods for predicting or diagnosing a FUS1 immune related disorder in a subject comprising determining a level of FUS1 expression in the subject.

In one embodiment, a reduced level of FUS1 in the sample indicates that subject has or is at risk of developing a FUS1 related disorder.

In one embodiment, the methods comprise determining a level of IL15 in a subject or in a sample from a subject.

In another embodiment, reduced levels of IL15 in the sample indicate that the subject is at risk of developing, is suffering from or has a FUS1 related disorder.

The methods, in one embodiment, further comprise obtaining a sample from the subject. In a related embodiment, the sample is one or more of a tissue sample, blood, sputum, bronchial washings, biopsy aspirate, ductal lavage, mucous, urine, or other biological specimen from a subject.

In one embodiment, determining comprises an immunoassay. In a related embodiment, the determining comprises, for example, one or more of a quantitative immunoassay, e.g., Western blots or ELISAs, quantitative RT-PCR, or Northern blot.

In certain embodiments, the FUS1 immune related disorder is an autoimmune disease, anemia, hematopoietic tumor, a virus associated malignancy, inflammatory infiltration of vessels, glomerulonephritis, vascular tumor, circulating antibodies, vasculitis, lymphoma, or NK cell maturation defect.

According to one aspect, methods of treating, preventing or alleviating a FUS1 related disorder in a subject are provided. The methods comprise administering a therapeutically effective amount of an IL15 composition to the subject.

In one embodiment, the FUS1 related disorder is one or more of cancer, an autoimmune disease, anemia, hematopoietic tumor, a virus associated malignancy, inflammatory infiltrating of vessels, glomerulonephritis, or vascular tumor. In a related embodiment, the cancer is breast cancer, lung cancer, prostate cancer, ovarian cancer, brain cancer, liver cancer, prostate cancer, cervical cancer, colon cancer, renal cancer, skin cancer, head & neck cancer, bone cancer, esophageal cancer, bladder cancer, uterine cancer, lymphatic cancer, stomach cancer, pancreatic cancer or testicular cancer.

In one embodiment, the composition may be administered, for example, systemically, intratumorally, intravascularally, to a resected tumor bed, orally, or by inhalation. In one embodiment, the IL15 composition comprises a substantially purified IL15 polypeptide, or fragment or variant thereof, for example, a sequence as defined by SEQ ID NO: 15 or 16. See T. A. Waldmann and Y. Tagaya, “The multifaceted regulation of Interleukin-15 Expression and the role of this Cytokine in NK Cell Differentiation and Host Response to Intracellular Pathogens,” Annu. Rev. Immunol. 1999. 17:19-49

In another embodiment, the IL15 composition comprises an nucleic acid encoding an IL15 polypeptide or fragment or variant thereof. In a related embodiment, the IL15 composition is a IL15 expression construct.

Provided herein, according to one aspect, are transgenic non-human animals comprising a homozygous disruption of the FUS1 gene, the FUS1 gene is disrupted by insertion of a transgene within a FUS1 locus within the genome of the non-human animal, no detectable FUS1 protein is expressed, and the non-human animal exhibits a phenotype comprising one or more of NK cell maturation defect, vasculitis, glomerulonephritis, auto-antibody production, or blood cell abnormalities.

In one embodiment, the transgene is inserted into the FUS1 locus by homologous recombination.

In another embodiment, the insertion removes at least a portion of an endogenous FUS1 gene within the genome of the non-human animal, the portion comprising at least the promoter, one functional domain, the start codon, a NarI-HindIII fragment, the entire coding region, the first exon, the second exon, the first, second, and a portion of the third exon of the endogenous FUS1 gene.

In one embodiment, the insertion of the transgene results in the replacement of the portion of the endogenous FUS1 gene with a nucleic acid sequence encoding a selectable marker.

In another embodiment, the selectable marker is a neomycin resistance gene.

In certain embodiments, the transgenic non-human animal is a primate, mouse, dog, cat, sheep, horse, or rabbit.

Provided herein, in one aspect, are one or more cells isolated from the transgenic non-human animals provided herein.

In one aspect, provided herein are transgenic non-human animals whose genome comprises a disruption in an endogenous FUS1 gene comprising the nucleic acid sequence set forth in SEQ ID NO: 1, or a fragment or variant thereof, where the disruption is homozygous, the transgenic non-human animal lacks production of functional protein encoded by the nucleic acid sequence, and exhibits one or more of the following phenotypes phenotype comprising one or more of NK cell maturation defect, vasculitis, glomerulonephritis, auto-antibody production, or blood cell abnormalities.

According to one aspect, presented herein are methods of making a transgenic non-human animal comprising a homozygous disruption of the FUS1 gene, the method comprising:

(i) transfecting a plurality of non-human animal embryonic stem cells with a nucleic acid comprising a FUS1 gene that is disrupted by insertion of a selectable marker;

(ii) selecting for transgenic embryonic stem cells that have incorporated the nucleic acid into their genome; and

(iii) introducing at least one of the transgenic embryonic stem cells into an embryo to produce a chimeric non-human animal comprising at least one of the transgenic embryonic stem cells.

The methods may further comprise breeding the chimeric non-human animal with a wild type non-human animal to obtain F1 progeny that are heterozygous for a disrupted FUS1 gene. The methods may also further comprise breeding a male non-human animal of the F1 progeny with a female non-human animal of the F1 progeny to obtain F2 progeny that are homozygous for the disrupted FUS1 gene; the non-human animal comprises a homozygous disruption of the FUS1 gene, no detectable FUS1 protein is expressed, and further exhibits a phenotype comprising one or more of NK cell maturation defect, vasculitis, glomerulonephritis, auto-antibody production, or blood cell abnormalities.

According to one aspect, methods of making a transgenic non-human animal comprising a homozygous disruption of the FUS1 gene are presented. The method comprise:

(i) transfecting a plurality of non-human animal embryonic stem cells with a nucleic acid comprising a FUS1 gene that is disrupted by insertion of a selectable marker;

(ii) selecting for transgenic embryonic stem cells that have incorporated the nucleic acid into their genome;

(iii) introducing at least one of the transgenic embryonic stem cells into an embryo to produce a chimeric non-human animal comprising at least one of the transgenic embryonic stem cells;

(iv) breeding the chimeric non-human animal with a wild type non-human animal to obtain F1 progeny that are heterozygous for a disrupted FUS1 gene; and

(v) breeding a male non-human animal of the F1 progeny with a female non-human animal of the F1 progeny to obtain F2 progeny that are homozygous for the disrupted FUS1 gene; the non-human animal comprises a homozygous disruption of the FUS1 gene, no detectable FUS1 protein is expressed, and further exhibits a phenotype comprising one or more of NK cell maturation defect, vasculitis, glomerulonephritis, auto-antibody production, or blood cell abnormalities.

Another aspect provides cells obtained from the transgenic non-human animal of certain aspects and embodiments described herein, in which the cell lacks production of functional protein encoded by the nucleotide sequence comprising SEQ ID NO: 1 or a fragment or variant thereof.

Also provided herein are methods of producing a transgenic non-human animal comprising a disruption in an endogenous FUS1 gene comprising the nucleic acid sequence set forth in SEQ ID NO: 1, comprising:

(a) introducing a targeting construct capable of disrupting the endogenous FUS1 gene comprising the nucleotide sequence set forth in SEQ ID NO: 1 into a non-human animal embryonic stem cell;

(b) selecting a murine embryonic stem cell that has undergone homologous recombination;

(c) introducing the murine embryonic stem cell into a blastocyst;

(d) implanting the resulting blastocyst into a pseudopregnant non-human animal, the non-human animal gives birth to a chimeric non-human animal; and

(e) breeding the chimeric non-human animal to produce the transgenic non-human animal, where the disruption is homozygous, the transgenic non-human animal lacks production of functional protein encoded by the nucleic acid sequence set forth in SEQ ID NO: 1, and exhibits at least one of the following phenotypes: kinky tail, low body weight or short body length, relative to a wild-type non-human animal.

In one embodiment, the non-human animal embryonic stem cell is murine, porcine, or primate.

In one aspect, methods of producing a non-human animal whose genome is homozygous for a disrupted FUS1 gene are provided, such that the non-human animal has no detectable FUS1. The method comprises:

(a) providing a gene encoding an altered form of FUS1 designed to target the FUS1 gene of non-human animal embryonic stem (ES) cells, the form comprises a disruption such that no detectable FUS1 is produced;

(b) introducing the gene encoding an altered form of FUS1 into non-human animal ES cells;

(c) selecting ES cells in which the altered gene encoding an altered form of FUS1 has disrupted the wild-type FUS1 gene;

(d) injecting the ES cells from step (c) into non-human animal blastocysts;

(e) implanting the blastocysts from step (d) into a pseudopregnant non-human animal; and

(f) allowing the blastocysts to develop into embryos and allowing the embryos to develop to term in order to produce a non-human animal homozygous for a disrupted FUS1 gene.

Other embodiments of the invention are disclosed infra.

### BRIEF DESCRIPTION OF THE DRAWINGS

FIG. 1 depicts the expression pattern of Fus1 in mouse tissues and human blood cells. (A) Northern blot analysis of Fus1 in selected mice tissues (B) PCR analysis on cDNA isolated from lymphoid and other mice tissues (MTCII cDNA panel “Clontech”). 1-liver; 2-uterus; 3-stomach; 4-eye; 5-prostate; 6-smooth muscle; 7-thymus; 8-lymph node; 9-bone marrow. (C) PCR analysis of Fus1, IL-15 and IL-2 expression in various human blood cells. Placenta cDNA was used as a control supplied by the manufacturer.

FIG. 2 depicts the localization of FUS1 shows that FUS1 is localized in mitochondria. (A) The Fus1/FLAG in transfected 293T cells was stained with the monoclonal anti-FLAG M2 antibody and visualized with Alexa⁵⁹⁴ conjugated (red) anti-mouse antibodies. Cells were stained simultaneously with polyclonal antibodies against either PDI, an ER marker (upper panel) or cytochrome c, a mitochondria marker (bottom panel), and then visualized with Alexa⁴⁸⁸ (green)-conjugated anti-rabbit IgG antibodies. (B) 293T cells transfected with Fus1/FLAG construct were fractionated (see Methods section) and aliquots of nuclear (N), mitochondrial (M) and cytoplasmic (C) fractions were run on 8-16% SDS-PAGE, transferred to a nitrocellulose membrane, and Western Blots were performed with anti-FLAG (Fus1 protein) or cytochrome c Abs.

FIG. 3 pictorially show the disruption of the Fus1 gene. (A) Diagram showing the Fus1 locus, the targeting construct, and the recombined mutant allele. Filled boxes indicate all three exons; the translation initiation site (arrow) and stop-codon (asterisk) are marked. (B) Southern analysis of EcoRI-digested DNA from Fus1^{-/-} (lanes 1 and 2), WT (lanes 3 and 4) and Fus1^{+/-} (lanes 5 and 6) mice. (C) Northern analysis of brain polyA RNA showing Fus1 mRNA from WT (lane 1), Fus1^{+/-} (lane 2) and Fus1^{-/-} (lane 3) mice. The blot was re-probed to detect the transcript of the Hyal2 gene adjacent to Fus1 gene that also served as a loading control. The membrane was stained before blotting with methylene blue to show equal RNA loading.

FIG. 4 depicts that Fus1-deficient mice develop signs of autoimmune disease. (A) Presence of circulating autoantibodies to nuclear antigens in 8-10 month old Fus1-deficient mice. Nuclear extracts from wild-type thymocytes were analyzed by Western blot using sera from WT, Fus1^{+/-} and Fus1^{-/-} mice. (B) Vasculitis in Fus1^{+/-} and Fus1^{-/-} 12 mo old mice. Panel A. Localized severe inflammation of a coronary artery (hematoxylin and eosin staining). Low magnification. Panel B. Higher magnification of the coronary lesion showing whorled inflammatory infiltrate in the artery wall and leukocytes (L, arrow) adhering to the endothelium (E). Panel C. Unaffected coronary artery. Panels D and E. Necrotizing arteritis in pancreas. Typical lesions, showing massive infiltration of the entire vessel and subendotelial fibrinoid necrosis (arrow) (Panel D) (H&E stain) (Panel E) Mason Trichrome stain. Panel F. Normal pancreatic vessels in WT mice. (H&E stain). Panel G. Arteritis in thyroid (low magnification). Panels H-J. Arteritis in omentum (Panel H) H&E stain, low magnification. (Panels I, J) High magnification, stained for elastin and connective tissue fibrils with elastin van Gieson's stain. In panel I, note that the smooth muscle layer becomes expanded and progressively disorganized. Inflammatory infiltrate, filling much of the field, surrounds the vessel. In panel J, note that the entirety of both layers of elastic tissue that surround the significantly expanded and disorganized muscle layer is disturbed. Also, loose connective tissue of the tunica adventia (red staining) that should closely surround the normal vessel is dispersed by the infiltration around the inflamed vessel. Panels K and L. Representative histological appearance of advanced glomerular lesions in Fus1^{-/-} mouse. H&E staining (Panel K), PAS-staining (Panel L). The glomerular lesions are characterized by the voluminous deposition of PAS-positive material, which nearly occlude the capillary lumens.

FIG. 5 shows Ig production in Fus1^{-/-} mice. (A) Sera from five 12-month old Fus1-deficient mice and controls were assayed for total Ig levels by ELISA. Data points represent the titers of individual mice; the filled circles are WT and open circles Fus1^{-/-}. The line represents the geometric mean for each group. (B) Ten Fus1^{-/-} and nine WT mice were immunized with TNP-KLH/Alum i.p., and serum was collected at the indicated time points. Serum levels of Ag-specific IgG1 and IgM were assayed by ELISA; data points represent the mean □SEM of the ELISA titers. The limit of detection of the assays was 200.

FIG. 6 shows altered NK cell maturation in Fus1^{-/-} mice is rescued after IL-15 stimulation. Representative FACS analysis of CD94 and Ly49G expression on CD3⁻DX5⁺ NK cells in WT and Fus1^{-/-} mice. (A) NK cells from untreated mice. (B) NK cells from mice treated with IL-15. Numbers indicate the percentage of the mature CD94⁺Ly49G⁺ NK cell subpopulation. The block in NK cell maturation i.e. the decreased percentage of CD94⁺Ly49G⁺ NK cells observed in Fus1^{-/-} mice (A) is corrected after IL-15 stimulation (B) as the two NK cell compartments show a similar CD94/Ly49G pattern.

FIG. 7 show Fus1-deficient mice have decreased expression level of IL-15. (A) Side-by-side hybridization of SuperArray membranes with cDNA probe generated on mRNAs isolated from the bone marrow or spleen of Fus1^{-/-} and WT mice. IL-15 cytokine mRNA levels were consistently decreased in Fus1^{-/-} mice (arrow). (B) Quantitative RT-PCR on the bone marrow mRNAs from three sets of Fus1^{-/-} and WT mice. Actin was used as a control for equal amount of cDNA in PCR reactions.

### DETAILED DESCRIPTION OF THE INVENTION

Disclosed herein are methods, systems, and transgenic animals useful for screening, diagnosing, and treating Fus1 related disorders. Further disclosed herein are novel methods for inhibiting cellular proliferation disorders as well as immune system disorders. As described herein, Fus1-deficient mice are viable, fertile, and demonstrate a complex immunological phenotype. Animals with a disrupted Fus1 gene developed signs of autoimmune disease, such as vasculitis, glomerulonephritis, anemia, circulating autoantibodies, and showed an increased frequency of spontaneous vascular and hematopoietic tumors. Fus1 null mice demonstrated a consistent defect in NK cell maturation that correlated with changes in the expression of the IL-15. Injection of IL-15 into Fus1 knockout mice completely rescued the NK cell maturation defect.

As used herein, “inhibiting cellular proliferation” includes slowing cellular proliferation as well as completely eliminating cellular proliferation. The slowing or eliminating may be by apoptotic cell death.

As used herein, “immune system cell” refers to, for example, NK cells, T cells, B cells, activated T cells, and activated B cells.

“Providing a Fus1 polypeptide,” refers to obtaining, by for example, buying or making the Fus1 polypeptides. They FUS1 polypeptides may be made by any known or later developed biochemical techniques. For example, the polypeptides may be obtained from cultured cells. The cultured cells, for example, may comprise an expression construct comprising a nucleic acid segment encoding FUS1.

A “pseudogene” as used herein, refers to a type of gene sequence found in the genomes, typically, of eucaryotes, where the sequence closely resembles a known functional gene, but differs in that the pseudogene is non-functional. For example, the pseudogene sequence may contain several stop codons in what would correspond to an open reading frame in the functional gene. Pseudogenes can also have deletions or insertions relative to their corresponding functional gene. If, for example, in a genome there is a functional gene and a related pseudogene, the functional gene is considered to be a single-copy gene (accordingly, the pseudogene is considered to be single-copy as well). Promoters, as used herein, refer to regulatory sequence of nucleic acid, for example, a FUS1 nucleic acid segment is referred to as being under the control of a promoter (e.g, a heterologous promoter). Polypeptides are produced from expression constructs, for example, comprising a nucleic acid segment encoding FUS1 under the control of a promoter. Expression constructs, may be, for example, viral or non-viral. For example, viral expression constructs include adenovirus, retrovirus, adeno-associated virus, herpesvirus, vaccinia virus or polyoma virus. For example, useful methods are described in Current Protocols in Protein Science, Chapter 5 Production of Recombinant Proteins (2005) by John Wiley & Sons, Inc.; and Current Protocols in Molecular Biology Chapter 16, Protein Expression (2005,) John Wiley & Sons, Inc.

Cells and/or subjects may be treated and/or contacted with one or more anti-proliferative treatments including, surgery, chemotherapy, radiotherapy, gene therapy, immune therapy or hormonal therapy, or other therapy recommended or proscribed by self or by a health care provider.

As used herein, “treating, preventing or alleviating a FUS1 immune related disorder” refers to the prophylactic use of FUS1 therapeutics and the use after diagnosis of a FUS1 related disorder.

As used herein a “FUS1 related disorder” is one or more of an autoimmune disease, anemia, hematopoietic tumor, a virus associated malignancy, inflammatory infiltrating of vessels, glomerulonephritis, vascular tumor, circulating antibodies, vasculitis, lymphoma, or NK cell maturation defect. Fus1 related disorders may also include wherein the cancer is breast cancer, lung cancer, prostate cancer, ovarian cancer, brain cancer, liver cancer, prostate cancer, cervical cancer, colon cancer, renal cancer, skin cancer, head & neck cancer, bone cancer, esophageal cancer, bladder cancer, uterine cancer, lymphatic cancer, stomach cancer, pancreatic cancer or testicular cancer.

As used herein a “reduced level” of FUS1 polypeptide, or fragments or variants thereof refers to a lower than average, expected or an actual lower value of expression for a particular cell or subject.

“Substantially purified” when used in the context of a FUS1 polypeptide, or fragment or variant thereof that are at least 60% free, preferably 75% free and more preferably 90% free from other components with which they are naturally associated. An “isolated polynucleotide” is, therefore, a substantially purified polynucleotide.

The term “subject” includes organisms which are capable of suffering from a FUS1 related disorder or who could otherwise benefit from the administration of a compound or composition of the invention, such as human and non-human animals. Preferred human animals include human patients suffering from or prone to suffering from a Fus1 related disorder or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

A method for “predicting or diagnosing” as used herein refers to a clinical or other assessment of the condition of a subject based on observation, testing, or circumstances.

“Determining a level of FUS1 expression” may be by any now known or hereafter developed assay or method of determining expression level, for example, immunological techniques, PCR techniques, immunoassay, quantitative immunoassay, Western blot or ELISA, quantitative RT-PCR, and/or Northern blot.

A sample or samples may be obtained from a subject, for example, by swabbing, biopsy, lavage or phlebotomy. Samples include tissue samples, blood, sputum, bronchial washings, biopsy aspirate, or ductal lavage.

“Therapeutically effective amount” as used herein refers to an amount of an agent which is effective, upon single or multiple dose administration to the cell or subject, in or in prolonging the survivability of the patient with such a disorder beyond that expected in the absence of such treatment.

An “IL15 composition” as used herein refers to a substantially purified IL15 polypeptide, or fragment or variant thereof, a nucleic acid encoding an IL15 polypeptide or fragment or variant thereof, and/or an IL15 expression construct.

Compositions described herein may be administered, for example, systemically, intratumorally, intravascularally, to a resected tumor bed, orally, or by inhalation.

As used herein, a “transgenic non-human animal” refers to a non-human animal with a heterozygous or homozygous disruption of the FUS1 gene. The FUS1 gene is disrupted, for example, by insertion of a transgene within a FUS1 locus within the genome of the non-human animal. Upon insertion, either a lesser amount or no detectable FUS1 protein is expressed and the non-human animal exhibits a phenotype comprising one or more of NK cell maturation defect, vasculitis, glomerulonephritis, auto-antibody production, or blood cell abnormalities.

The transgene or knock-out construct is inserted into the FUS1 locus, for example, by homologous recombination. The insertion removes at least a portion of an endogenous FUS1 gene within the genome of the non-human animal, the portion is, for example, one or more of the promoter, one functional domain, the start codon, a NarI-HindIII fragment, the entire coding region, the first exon, the second exon, the first, second, and a portion of the third exon of the endogenous FUS1 gene. In certain embodiments, the insertion of the transgene results in the replacement of the portion of the endogenous FUS1 gene with a nucleic acid sequence encoding a selectable marker. In certain embodiments, the selectable marker is a neomycin resistance gene.

The transgenic non-human animal may be a primate, mouse, dog, cat, sheep, horse, rabbit or other non-human animal.

Cells may be isolated and cultured from the transgenic non-human animals. The cells may be used in, for example, primary cultures or established cultures.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Boil. Chem. 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule, which is comprised of segments of DNA joined together by means of molecular biological techniques.

As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, an oligonucleotide having a nucleotide sequence encoding a gene refers to a DNA sequence comprising the coding region of a gene or in other words the DNA sequence, which encodes a gene product. The coding region may be present in either a cDNA or genomic DNA form. Suitable control elements such as enhancers/promoters, splice junctions, polyadenylation signals, etc., may be placed in close proximity to the coding region of the gene if needed to permit proper initiation of transcription and/or correct processing of the primary RNA transcript. Alternatively, the coding region utilized in the vectors of the present invention may contain endogenous enhancers/promoters, splice junctions, intervening sequences, polyadenylation signals, etc., or a combination of both endogenous and exogenous control elements.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which directs the degrading mechanism to other similar RNA sequences. Alternatively, RNAi can be initiated by the hand of man, for example, to silence the expression of target genes.

By “reduce or inhibit” is meant the ability to cause an overall decrease preferably of 20% or greater, more preferably of 50% or greater, and most preferably of 75% or greater, in the level of protein or nucleic acid, detected by the aforementioned assays (see “expression”), as compared to samples not treated with antisense nucleotide oligomers or dsRNA used for RNA interference. A siRNA having a “sequence sufficiently complementary to a target mRNA sequence to direct target-specific RNA interference (RNAi)” means that the ss-siRNA has a sequence sufficient to trigger the destruction of the target mRNA by the RNAi machinery or process.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

An RNAi agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

By “small interfering RNAs (siRNAs)” (also referred to in the art as “short interfering RNAs”) is meant an isolated RNA molecule comprising between about 10-50 nucleotides (or nucleotide analogs), which is capable of directing or mediating RNA interference. The siRNA is preferably greater than 10 nucleotides in length, more preferably greater than 15 nucleotides in length, and most preferably greater than 19 nucleotides in length that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule. siRNA includes any form of dsRNA (specifically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 21 to 23 nt RNA or internally (at one or more nucleotides of the RNA). In a preferred embodiment, the RNA molecules contain a 3′ hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNA interference (RNAi). RNAi agents of the present invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymidine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides. (Brummelkamp et al., Science 296:550-553 (2002); Lee et al, (2002). supra; Miyagishi and Taira, Nature Biotechnol. 20:497-500 (2002); Paddison et al. (2002), supra; Paul (2002), supra; Sui (2002) supra; Yu et al. (2002), supra).

siRNAs also include “single-stranded small interfering RNA molecules.” “Single-stranded small interfering RNA molecules” (“ss-siRNA molecules” or “ss-siRNA”). ss-siRNA is an active single stranded siRNA molecule that silences the corresponding gene target in a sequence specific manner. Preferably, the ss-siRNA molecule has a length from about 10-50 or more nucleotides. More preferably, the ss-siRNA molecule has a length from about 19-23 nucleotides. In addition to compositions comprising ss-siRNA molecules other embodiments of the invention include methods of making said ss-siRNA molecules and methods (e.g., research and/or therapeutic methods) for using said ss-siRNA molecules. As used herein, the term “specifically hybridizes” or “specifically detects” refers to the ability of a nucleic acid molecule to hybridize to at least approximately 6 consecutive nucleotides of a sample nucleic acid.

A “target gene” is a gene whose expression is to be selectively inhibited or “silenced,” for example Fus1. This silencing is achieved by cleaving the mRNA of the target gene by an siRNA that is created from an engineered RNA precursor by a cell's RNAi system. One portion or segment of a duplex stem of the RNA precursor is an anti-sense strand that is complementary, e.g., fully complementary, to a section of about 18 to about 40 or more nucleotides of the mRNA of the target gene.

Embodiments of the invention include the use of the ES cell lines derived from the transgenic zygote, embryo, blastocyst or non-human animal to treat human and non-human animal diseases. Methods include implanting ES cells into an organ, for example, the brain, liver, heart, kidney, pancreas, skin, and the like, and allowing the cells to develop into the organ tissue. For example, the ES cell lines may be implanted into the brain of a human suffering from Parkinson's, or into the pancreas of a diabetic patient, and the like to treat the condition. In addition, embryonic stem cells transduced with disease-causing gene mutations as provided herein, can provide an in vitro system to investigate disease pathogenesis and to test potential therapeutic strategies.

Transgenic non-human animals also include those whose genome comprises a disruption in an endogenous FUS1 gene comprising the nucleic acid sequence set forth in SEQ ID NO: 1, or a fragment or variant thereof, wherein where the disruption is homozygous, the transgenic non-human animal lacks production of functional protein encoded by the nucleic acid sequence, and exhibits one or more of the following phenotypes comprising one or more of NK cell maturation defect, vasculitis, glomerulonephritis, auto-antibody production, or blood cell abnormalities.

Detecting expression of a gene or protein includes examining the cell or cells of the transgenic zygote, embryo, blastocyst, fetus, or transgenic non-human animal cells for the integration of the transgene and/or the expression of the gene. The integration of the gene may be detected, for example, by Southern blot or Polymerase chain reaction (PCR) may be performed with primer sets that cover the gene. Expression of the gene may be examined in transgenic non-human animals, for example, in their hair, blood, umbilical cord, placenta, cultured lymphocytes, buccal epithelial cells, and urogenital cells passed in urine. Expression may also be examiner by extracting total RNA for reverse transcription followed by PCR amplification (RT-PCR) with primer sets specific for the gene or protein.

Methods of making a transgenic non-human animal comprising a heterozygous or homozygous disruption of the FUS1 gene are presented herein. The methods comprise:

(i) transfecting a plurality of non-human animal embryonic stem cells with a nucleic acid comprising a FUS1 gene that is disrupted by insertion of a selectable marker;

(ii) selecting for transgenic embryonic stem cells that have incorporated the nucleic acid into their genome;

(iii) introducing at least one of the transgenic embryonic stem cells into an embryo to produce a chimeric non-human animal comprising at least one of the transgenic embryonic stem cells;

(iv) breeding the chimeric non-human animal with a wild type non-human animal to obtain F1 progeny that are heterozygous for a disrupted FUS1 gene; and optionally

(v) breeding a male non-human animal of the F1 progeny with a female non-human animal of the F1 progeny to obtain F2 progeny that are homozygous for the disrupted FUS1 gene; wherein the non-human animal comprises a homozygous disruption of the FUS1 gene, wherein no detectable FUS1 protein is expressed, and further exhibits a phenotype comprising one or more of NK cell maturation defect, vasculitis, glomerulonephritis, auto-antibody production, or blood cell abnormalities.

Cells obtained from the transgenic non-human animals described herein may be obtained by taking a sample of a tissue of the animal. The cells may then be cultured. The cells preferably lack production of functional protein encoded by the nucleotide sequence comprising SEQ ID NO: 1 or a fragment or variant thereof.

Methods of producing a transgenic non-human animal having a disruption in an endogenous FUS1 gene of the nucleic acid sequence set forth in one or more of SEQ ID NO: 1-13, comprise:

a) introducing a targeting construct capable of disrupting the endogenous FUS1 gene comprising the nucleotide sequence set forth in one or more of SEQ ID NO: 1-13 into a non-human animal embryonic stem cell;

b) selecting a murine embryonic stem cell that has undergone homologous recombination;

(c) introducing the murine embryonic stem cell into a blastocyst;

(d) implanting the resulting blastocyst into a pseudopregnant non-human animal, wherein the non-human animal gives birth to a chimeric non-human animal; and

(e) breeding the chimeric non-human animal to produce the transgenic non-human animal, wherein where the disruption is homozygous, the transgenic non-human animal lacks production of functional protein encoded by the nucleic acid sequence set forth in one or more of SEQ ID NO: 1-13, and exhibits at least one of the following phenotypes: a kinky tail, low body weight or short body length, relative to a wild-type non-human animal.

the non-human animal embryonic stem cell is murine, porcine, or primate.

Another method of producing a non-human animal whose genome is heterozygous or homozygous for a disrupted FUS1 gene, such that the non-human animal has no detectable FUS1, the method comprising:

(a) providing a gene encoding an altered form of FUS1 designed to target the FUS1 gene of non-human animal embryonic stem (ES) cells, wherein the form comprises a disruption such that no detectable FUS1 is produced;

(b) introducing the gene encoding an altered form of FUS1 into non-human animal ES cells;

(c) selecting ES cells in which the altered gene encoding an altered form of FUS1 has disrupted the wild-type FUS1 gene;

(d) injecting the ES cells from step (c) into non-human animal blastocysts;

(e) implanting the blastocysts from step (d) into a pseudopregnant non-human animal; and

(f) allowing the blastocysts to develop into embryos and allowing the embryos to develop to term in order to produce a non-human animal homozygous for a disrupted FUS1 gene.

The present invention provides transgenic and chimeric non-human mammals comprising one or more functionally and structurally disrupted FUS1 alleles.

A “chimeric animal” includes some cells that lack the functional FUS1 gene of interest and other cells that do not have the inactivated gene. A “transgenic animal,” in contrast, is made up of cells that have all incorporated the specific modification, which renders the FUS1 gene inactive or otherwise altered. While a transgenic animal is typically capable of transmitting the mutant FUS1 gene to its progeny, the ability of a chimeric animal to transmit the mutation depends upon whether the inactivated gene is present in the animal's germ cells. The modifications that inactivate or otherwise alter the FUS1 gene can include, for example, insertions, deletions, or substitutions of one or more nucleotides. The modifications can interfere with transcription of the gene itself, with translation and/or stability of the resulting mRNA, or can cause the gene to encode an inactive or otherwise altered FUS1 polypeptide, e.g., a FUS1 polypeptide with modified properties. In particular, the present transgenic and chimeric animals can lack coding sequences for one or more components of a FUS1 polypeptide, such as the kinase domain, heterologous protein binding domains, etc. Such transgenes can thus eliminate any one or more codons within an endogenous FUS1 allele. In a preferred embodiment, a transgenic animal has an allele that lacks at least 20, 30, 40, or more codons of the full-length protein. Further, a transgenic animal can lack non-coding sequences that are required for FUS1 expression or function, such as 5′ or 3′ regulatory sequences. For example, at least the promoter, one functional domain, the start codon, a NarI-HindIII fragment, the entire coding region, the first exon, the second exon, the first, second, and a portion of the third exon of the endogenous FUS1 gene.

The claimed methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, C A, Ed., Transgenic Animal Technology: A Laboratory Handbook, Academic Press, 1994.

In certain embodiments, transgenic mice will be produced as described in Thomas et al. (1999) Immunol., 163:978-84; Kanakaraj et al. (1998) J. Exp. Med., 187:2073-9; or Yeh et al. (1997) Immunity 7:715-725.

Typically, a modified FUS1 gene is introduced, e.g., by homologous recombination, into embryonic stem cells (ES), which are obtained from preimplantation embryos and cultured in vitro. See, e.g., Hooper, M L, Embryonal Stem Cells: Introducing Planned Changes into the Animal Germline (Modeem Genetics, v. 1), Int'. Pub. Distrib., Inc., 1993; Bradley et al. (1984) Nature, 309, 255-258. Subsequently, the transformed ES cell is combined with a blastocyst from a non-human animal, e.g., a mouse. The ES cells colonize the embryo and in some embryos form the germ line of the resulting chimeric animal. See, Jaenisch (1988) Science, 240:1468-1474. Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature, 385: 810-813.

Other methods for obtaining a transgenic or chimeric animal having a mutant FUS1 gene in its genome is to contact fertilized oocytes with a vector that includes a polynucleotide that encodes a modified, e.g., inactive, FUS1 polypeptide. In some animals, such as mice, fertilization is typically performed in vivo and fertilized ova are surgically removed. In other animals, particularly bovines, it is preferably to remove ova from live or slaughterhouse animals and fertilize the ova in vitro. See DeBoer et al., WO 91/08216. In vitro fertilization permits the modifications to be introduced into substantially synchronous cells.

Oocytes for use in the invention include oocytes at any state of maturity that will allow fertilization, preferably, ooctyes in metaphase II stage of meiotic cell division, e.g., oocytes arrested in metaphase II, a telophase stage of meiotic cell division, e.g., telophase I or telophase II. Oocytes in metaphase II contain one polar body, whereas oocytes in telophase can be identified by the absence of a germinal vesicle and polar body or based on the presence of a protrusion of the plasma membrane from the second polar body up to the formation of a second polar body. In addition, oocytes in metaphase II can be distinguished from oocytes in telophase II based on biochemical and/or developmental distinctions. For example, oocytes in metaphase II can be in an arrested state, whereas oocytes in telophase are in an activated state. Preferably, the oocyte is a non-human primate.

Oocytes can be obtained or recovered at various times at a various stages of development or maturation during a non-human animals reproductive cycle. For example, at given times during the reproductive cycle, a significant percentage of the oocytes, e.g., about 55%, 60%, 65%, 70%, 75%, 80% or more, are oocytes in prophase or telophase I. Such oocytes at various stages of the cell cycle can be obtained or recovered from the non-human primate and then induced in vitro to enter a particular stage of meiosis.

Oocytes can also be collected or recovered from a female non-human primate during superovulation. Briefly, oocytes can be recovered surgically by inserting a needle into each ovarian follicle and aspirating the follicular content. Alternately, oocytes that have been ovulated can be recovered by flushing the oviduct of the female donor. In this case, the female donor has either ovulated during a natural cycle or has been subjected to a modified superovulation protocol. Fertilized oocytes are also useful and can be obtained or recovered from the oviducts of mated non-human animals. Such protocols are well known in the art and one of skill in the art, having the benefit of this disclosure would know how to effect superovulation in a female non-human animal or recover oocytes from mated females. Methods of inducing superovulation in non-human animals and the collection of oocytes is described in the examples herein.

The method includes contacting the oocyte with sperm under conditions that permit the fertilization of the oocyte to produce an embryo. Fertilizing the oocyte to produce a zygote having zygotic pronuclei may be done by intracytoplasmic sperm injection, sperm incubation, or the like. These techniques are described in Ouhibi et al.

In preferred embodiments, the genetic construct is preferably introduced into a single-cell zygote. Such introduction may be achieved by pronuclear injection or microinjection (Wang, et al. Molecular Reproduction and Development (2002) 63:437-443), cytoplasmic injection or microinjection (Page, et al. Transgenic Res (1995) 4(6):353-360), retroviral infection (e.g., Lebkowski, et al. Mol Cell Biol (1988) 8(10):3988-3996), or electroporation (“Molecular Cloning: A Laboratory Manual. Second Edition” by Sambrook, et al. Cold Spring Harbor Laboratory: 1989). Introduction may also be by chemical assistance, for example, by lysosomal vesical packaging or other similar technique. For injection or microinjection and electroporation protocols, the introduced DNA may comprise linear or circular DNA, as prepared from the vectors or constructs of the invention. This introduction of the genetic construct and the AAV Rep protein should not interfere with early embryo development and should result in gene expression. According to further methods, the zygote is allowed to further develop into, for example, a pre-implantation embryo suitable for implantation into a recipient female for fetal development. The genetic construct may be introduced, for example, into the male pronuclei, the female pronuclei, or both the male and female pronuclei.

Other references for introduction of knock-out constructs into embryonic cells are known in the art. See, for example, “Transgenic Animal Technology: A Laboratory Handbook,” C. A. Pinkert, editor, Academic Press, 2002, 2nd edition, 618 pp.; “Mouse Genetics and Transgenics: A Practical Approach,” I. J. Jackson and C. M. Abbott, editors, Oxford University Press, 2000, 299 pp.; “Transgenesis Techniques: Principles and Protocols,” A. R. Clarke, editor, Humana Press, 2001, 351 pp., Briskin et al. (1991) Proc. Natl. Acad. Sci. USA, 88:1736-1740; Pfeifer et al. (2002), Proc. Natl. Acad. Sci. USA, 99:2140-2145; Houdebine and Chourrout (1991) Experientia, 47:891-897, Carver, et al., Bio/Technology 11:1263-1270, 1993; Carver et al., Cytotechnology 9:77-84, 1992; Clark et al, Bio/Technology 7:487-492, 1989; Simons et al., Bio/Technology 6:179-183, 1988; Swanson et al., Bio/Technology 10:557-559, 1992; Velander et al., Proc. Natl. Acad. Sci. USA 89:12003-12007, 1992; Hammer et al., Nature 315:680-683, 1985; Krimpenfort et al., Bio/Technology 9:844-847, 1991; Ebert et al., Bio/Technology 9:835-838, 1991; Simons et al., Nature 328:530-532, 1987; Pittius et al., Proc. Natl. Acad. Sci. USA 85:5874-5878, 1988; Greenberg et al., Proc. Natl. Acad. Sci. USA 88:8327-8331, 1991; Whitelaw et al., Transg. Res. 1:3-13, 1991; Gordon et al., Bio/Technology 5:1183-1187, 1987; Grosveld et al., Cell 51:975-985, 1987; Brinster et al., Proc. Natl. Acad. Sci. USA 88:478-482, 1991; Brinster et al., Proc. Natl. Acad. Sci. USA 85:836-840, 1988; Brinster et al., Proc. Natl. Acad. Sci. USA 82:4438-4442, 1985; Al-Shawi et al., Mol. Cell. Biol. 10(3):1192-1198, 1990; Van Der Putten et al., Proc. Natl. Acad. Sci. USA 82:6148-6152, 1985; Thompson et al., Cell 56:313-321, 1989; Gordon et al., Science 214:1244-1246, 1981; and Hogan et al., Manipulating the Mouse Embryo: A Laboratory Manual (Cold Spring Harbor Laboratory, 2002), which are each incorporated herein by reference in their entirety.

One method further comprises, transferring the oocyte, zygote, blastocyst, or embryo into a hormonally synchronized non-human recipient animal (i.e., a female animal at the correct stage of the menstrual cycle to support embryo implantation and development or a female animal hormonally synchronized to stimulate early pregnancy). Methods of transfer include, embryo placement into the oviduct by laparoscopy or mini-laparotomy, and a non-surgical, trans-cervical approach of uterine deposition. Other acceptable methods of transfer include, cervical cannulation, and the like. In another preferred embodiment, the method comprises the step of allowing the transferred embryo/pregnancy to develop to term. Developing to term includes developing until the transgenic embryo would be viable outside of the uterus. In still another preferred embodiment, at least one transgenic offspring is identified from the offspring allowed to develop to term. Method to introduce nucleic acid into a cell useful in the method described herein include protein transduction techniques, (See “Transmembrane delivery of protein and peptide drugs by TAT-mediated transduction in the treatment of cancer,” Jehangir S. Wadia, Steven F. Dowdy, Advanced Drug Delivery Reviews, 57 (2005) 579-596) and viral methods, such as AAV and other viral vectors. Examples of protein transduction domains include, Drosophila homeotic transcription protein antennapedia (Antp), the herpes simplex virus structural protein VP22, and the human immunodeficiency virus 1 (HIV-1) transcriptional activator Tat protein.

The selection of embryos for transfer is normally based on developmental progression, presence of the appropriate number of nucleated blastomeres, absence of fragmentation and general appearance. Usually only the highest quality embryos are transferred. The ability to freeze embryos and conduct transfers when recipients are available is highly convenient because it supports the shipment of embryos to other facilities.

Methods may further include mating the transgenic non-human animal that develops from the transgenic embryo with a second non-human animal. The second non-human animal can be a normal non-human animal, a second non-human animal which develops from a transgenic embryo or is descended from a non-human animal which developed from a transgenic embryo or a second non-human animal developed from a transgenic embryo, or descended from a non-human animal which developed from a transgenic embryo, which was formed from genetic material from the same animal, an animal of the same genotype, or same cell line, which supplied the genetic material for the first non-human animal. In a preferred embodiment, a first transgenic non-human animal that develops from the transgenic embryo can be mated with a second transgenic non-human animal which developed from a transgenic embryo and which contains a different gene than the first transgenic non-human animal.

In one embodiment, the transgenic non-human animal is a male non-human animal. In other preferred embodiments the transgenic non-human animal is a female non-human animal.

According to other embodiments, the transgenic non-human animal oocyte, blastocyst, embryo, or offspring may be used as a model for a human disease.

In certain embodiments, the cells of the transgenic oocyte, zygote, blastocyst, or embryo are used to establish embryonic stem (ES) cell lines. Stem cells are defined as cells that have extensive proliferation potential, differentiate into several cell lineages, and repopulate tissues upon transplantation. The quintessential stem cell is the embryonic stem (ES) cell, as it has unlimited self-renewal and multipotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass of the blastocyst (Thomson, J. et al. 1995; Thomson, J. A. et al. 1998; Martin, G. R. 1981), or can be derived from the primordial germ cells from a post-implantation embryo (embryonic germ cells or EG cells). ES and EG cells have been derived from mouse, and more recently also from non-human animals and humans. When introduced into mouse blastocysts, ES cells can contribute to all tissues of the mouse (animal) (Orkin, S. 1998). Murine ES cells are therefore pluripotent. When transplanted in post-natal animals, ES and EG cells generate teratomas, which again demonstrates their multipotency. ES (and EG) cells can be identified by positive staining with the antibodies to stage-specific embryonic antigens (SSEA) 1 and 4.

Fertilized oocytes are typically cultured in vitro until a pre-implantation embryo is obtained containing about 16-150 cells. The 16-32 cell stage of an embryo is described as a morula, whereas pre-implantation embryos containing more than 32 cells are termed blastocysts. These embryos show the development of a blastocoel cavity, typically at the 64-cell stage. The presence of a desired FUS1 mutation in the cells of the embryo can be detected by methods known to those of skill in the art, e.g., Southern blotting, PCR, DNA sequencing, or other standard methods. Methods for culturing fertilized oocytes to the preimplantation stage are described, e.g., by Gordon et al. (1984) Methods Enzymol., 101:414; Hogan et al. Manipulation of the Mouse Embryo: A Laboratory Manual, C.S.H.L. N.Y. (1986) (mouse embryo); Hammer et al. (1985) Nature, 315: 680 (rabbit and porcine embryos); Gandolfi et al. (1987) J. Reprod. Fert., 81:23-28; Rexroad et al. (1988) J. Anim. Sci., 66:947-953 (ovine embryos) and Eyestone et al. (1989) J. Reprod. Fert., 85:715-720; Camous et al. (1984) J. Reprod. Pert., 72:779-785; and Heyman et al. (1987) Theriogenology, 27:5968 (bovine embryos). Pre-implantation embryos may also be stored frozen for a period pending implantation.

Pre-implantation embryos are transferred to an appropriate female resulting in the birth of a transgenic or chimeric animal, depending upon the stage of development when the transgene is integrated. Chimeric mammals can be bred to form true germline transgenic animals. Chimeric mice and germline transgenic mice can also be ordered from commercial sources (e.g., Deltagen, San Carlos, Calif.).

Other methods for introducing mutations into mammalian cells or animals include recombinase systems, which can be employed to delete all or a portion of a locus of interest. Examples of recombinase systems include, the cre/lox system of bacteriophage P1 (see, e.g., Gu et al. (1994) Science, 265:103-106; Terry et al. (1997) Transgenic Res., 6:349-356) and the FLP/FRT site specific integration system (see, e.g., Dymecki (1996) Proc. Natl. Acad. Sci. U.S.A., 93:6191-6196). In these systems, sites recognized by the particular recombinase are typically introduced into the genome at a position flanking the portion of the gene that is to be deleted. Introduction of the recombinase into the cells then catalyzes recombination which deletes from the genome the polynucleotide sequence that is flanked by the recombination sites. If desired, one can obtain animals in which only certain cell types lack the FUS1 gene of interest, e.g., by using a tissue specific promoter to drive the expression of the recombinase. See. e.g., Tsien et al. (1996) Cell. 87: 1317-26; Brocard et al. (1996) Proc. Natl. Acad. Sci. U.S.A., 93:10887-10890; Wang et al. (1996) Proc. Natl. Acad. Sci. U.S.A., 93:3932-6; Meyers et al. (1998) Nat. Genet., 18:13641).

The presence of any mutation in a FUS1 gene in a cell or animal can be detected using any method described herein, e.g., Southern blot, PCR, DNA sequencing, or using assays based on any FUS1-dependent cell or organismal property or behavior. See, e.g., Ausubel et al., supra.

RNAi Compositions for Targeting Fus1 mRNA

This invention is generally related to treatment and management of cancer by using the Fus1 gene and its products. One embodiment of this invention is directed to a method comprising contacting the cell with a compound that inhibits the synthesis or expression of Fus1 gene in an amount sufficient to cause such inhibition. Without being limited by theory, the inhibition is achieved through selectively targeting Fus1 DNA or mRNA, i.e., by impeding any steps in the replication, transcription, splicing or translation of the Fus1 gene. The sequence of Fus1 is disclosed in GenBank Accession No. (SEQ. ID NO. 1-13), the entirety of which is incorporated herein by reference.

RNAi can be a remarkably efficient process whereby double-stranded RNA (dsRNA) induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490). In mammalian cells, RNAi can be triggered by 21-nucleotide (nt) duplexes of small interfering RNA (siRNA) (Chiu et al. (2002), Mol. Cell., 10, 549-561; Elbashir et al. (2001), Nature, 411, 494-498), or by micro-RNAs (miRNA), functional small-hairpin RNA (shRNA), or other dsRNAs which are expressed in-vivo using DNA templates with RNA polymerase III promoters (Zeng et al. (2002), Mol. Cell, 9, 1327-1333; Paddison et al. (2002), Genes Dev., 16, 948-958; Lee et al. (2002), Nature Biotechnol., 20, 500-505; Paul et al. (2002), Nature Biotechnol., 20, 505-508; Tuschl, T. (2002), Nature Biotechnol., 20, 440-448; Yu et al. (2002), Proc. Natl. Acad. Sci. USA, 99(9), 6047-6052; McManus et al. (2002), RNA, 8, 842-850; Sui et al. (2002), Proc. Natl. Acad. Sci. USA, 99(6), 5515-5520.)

The present invention features “small interfering RNA molecules” (“siRNA molecules” or “siRNA”), methods of making said siRNA molecules and methods (e.g., research and/or therapeutic methods) for using said siRNA molecules. A siRNA molecule of the invention is a duplex consisting of a sense strand and complementary antisense strand, the antisense strand having sufficient complementary to a target mRNA to mediate RNAi. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, such as a target region that differs by at least one base pair between the wild type and mutant allele, e.g., a target region comprising the gain-of-function mutation, and the other strand is identical or substantially identical to the first strand. small interfering RNA molecules

In one embodiment, the expression of Fus1 is inhibited by the use of an RNA interference technique referred to as RNAi. RNAi allows for the selective knockdown of the expression of a target gene in a highly effective and specific manner. This technique involves introducing into a cell double-stranded RNA (dsRNA), having a sequence corresponding to the exon portion of the target gene. The dsRNA causes a rapid destruction of the target gene's mRNA. See, e.g., Hammond et al., Nature Rev Gen 2: 110-119 (2001); Sharp, Genes Dev 15: 485-490 (2001), both of which are incorporated herein by reference in their entireties.

Methods and procedures for successful use of RNAi technology are well-known in the art, and have been described in, for example, Waterhouse et al., Proc. Natl. Acad. Sci. USA 95(23): 13959-13964 (1998). The siRNAs of this invention encompass any siRNAs that can modulate the selective degradation of Fus1 mRNA.

The siRNAs of the invention include “double-stranded small interfering RNA molecules” (“ds-siRNA” and “single-stranded small interfering RNA molecules” (“ss-siRNA”), methods of making the siRNA molecules and methods (e.g., research and/or therapeutic methods) for using the siRNA molecules.

Similarly to the ds-siRNA molecules, the ss-siRNA molecule has a length from about 10-50 or more nucleotides. More preferably, the ss-siRNA molecule has a length from about 15-45 nucleotides. Even more preferably, the ss-siRNA molecule has a length from about 19-40 nucleotides. The ss-siRNA molecules of the invention further have a sequence that is “sufficiently complementary” to a target mRNA sequence to direct target-specific RNA interference (RNAi), as defined herein, i.e., the ss-siRNA has a sequence sufficient to trigger the destruction of the target mRNA by the RNAi machinery or process. The ss-siRNA molecule can be designed such that every residue is complementary to a residue in the target molecule. Alternatively, substitutions can be made within the molecule to increase stability and/or enhance processing activity of a said molecule. Substitutions can be made within the strand or can be made to residues at the ends of the strand. The 5′-terminus is, most preferably, phosphorylated (i.e., comprises a phosphate, diphosphate, or triphosphate group). The 3′ end of a siRNA may be a hydroxyl group in order to facilitate RNAi, as there is no requirement for a 3′ hydroxyl group when the active agent is a ss-siRNA molecule. Featured are ss-siRNA molecules wherein the 3′ end (i.e., C3 of the 3′ sugar) lacks a hydroxyl group (i.e., ss-siRNA molecules lacking a 3′ hydroxyl or C3 hydroxyl on the 3′ sugar (e.g., ribose or deoxyribose).

The siRNAs of this invention include modifications to their sugar-phosphate backbone or nucleosides. These modifications can be tailored to promote selective genetic inhibition, while avoiding a general panic response reported to be generated by siRNA in some cells. Moreover, modifications can be introduced in the bases to protect siRNAs from the action of one or more endogenous enzymes.

The siRNAs of this invention can be enzymatically produced or totally or partially synthesized. Moreover, the siRNAs of this invention can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although 100 percent sequence identity between the siRNA and the target region is preferred, it is not required to practice this invention. siRNA molecules that contain some degree of modification in the sequence can also be adequately used for the purpose of this invention. Such modifications include, but are not limited to, mutations, deletions or insertions, whether spontaneously occurring or intentionally introduced. Specific examples of siRNAs that can be used to inhibit the expression of Fus1 are described in detail in Example 5. The target RNA cleavage reaction guided by siRNAs is highly sequence specific. In general, siRNA containing a nucleotide sequences identical to a portion of the target gene are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition.

Moreover, not all positions of a siRNA contribute equally to target recognition. Mismatches in the center of the siRNA are most critical and essentially abolish target RNA cleavage. In contrast, the 3′ nucleotides of the siRNA do not contribute significantly to specificity of the target recognition. In particular, residue 3′ of the siRNA sequence which is complementary to the target RNA (e.g., the guide sequence) are not critical for target RNA cleavage.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions ×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17): 3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the siRNA and the portion of the target gene is preferred. Alternatively, the ss-siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50 degrees C. or 70 degrees C. hybridization for 12-16 hours; followed by washing). Additional preferred hybridization conditions include hybridization at 70 degrees C. in 1×SSC or 50 degrees C. in 1×SSC, 50% formamide followed by washing at 70 degrees C. in 0.3×SSC or hybridization at 70 degrees C. in 4×SSC or 50 degrees C. in 4×SSC, 50% formamide followed by washing at 67 degrees C. in 1×SSC. The hybridization temperature for hybrids anticipated to be less than 50 base pairs in length should be 5-10 degrees C. less than the melting temperature (Tm) of the hybrid, where Tm is determined according to the following equations. For hybrids less than 18 base pairs in length, Tm (degrees C.)=2(# of A+T bases)+4(# of G+C bases). For hybrids between 18 and 49 base pairs in length, Tm (degrees C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In a preferred aspect, the RNA molecules of the present invention are modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In an embodiment of the present invention the RNA molecule may contain at least one modified nucleotide analogue. The nucleotide analogues may be located at positions where the target-specific activity, e.g., the RNAi mediating activity is not substantially affected, e.g., in a region at the 5′-end and/or the 3′-end of the RNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications might be combined.

The nucleic acid compositions of the invention include both siRNA and siRNA derivatives as described herein. For example, cross-linking can be employed to alter the pharmacokinetics of the composition, for example, to increase half-life in the body. Thus, the invention includes siRNA derivatives that include siRNA having two complementary strands of nucleic acid, such that the two strands are crosslinked. The invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

### Expression Constructs

To deliver FUS1 sequences to cells, one may introduce a nucleic acid segment coding for FUS1 into an expression vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression vector” or “expression construct” refers to a vector containing a nucleic acid sequence or “cassette” coding for at least part of a gene product capable of being transcribed and “regulatory” or “control” sequences, which refer to nucleic acid sequences necessary for the transcription and possibly translation of an operably linked coding sequence in a particular host cell. In addition to control sequences that govern transcription and translation, expression vectors may contain nucleic acid sequences that serve other functions as well and are described infra.

As used herein, the term “promoter/enhancer” denotes a segment of DNA which contains sequences capable of providing both promoter and enhancer functions (i.e., the functions provided by a promoter element and an enhancer element, see above for a discussion of these functions). Promoters and enhancers consist of short arrays of DNA sequences that interact specifically with cellular proteins involved in transcription (Maniatis et al., Science 236:1237, 1987).

The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of the gene is directed by the linked enhancer/promoter.

Promoters and enhancers may bind to specific factors, which increase the rate of activity from the promoter or enhancer. The term “factor” refers to a protein or group of proteins necessary for the transcription or replication of a DNA sequence. For example, SV40 T antigen is a replication factor necessary for the replication of DNA sequences containing the SV40 origin of replication. For example, transcription factors are proteins that bind to regulatory elements such as promoters and enhancers and facilitate the initiation of transcription of a gene. The selection of a particular promoter and enhancer depends on what cell type is to be used to express the protein of interest. Some eukaryotic promoters and enhancers have a broad host range while others are functional in a limited subset of cell types (Voss et al., Trends Biochem. Sci., 11:287, 1986; and Maniatis et al., supra., 1987.

The phrases “operatively positioned,” “operatively linked,” “under control,” and “under transcriptional control” mean that a promoter is in a correct functional location and orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence. Together, an appropriate promoter or promoter/enhance combination, and a gene of interest, comprise an expression cassette.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Such promoters may be used to drive β-galactosidase expression for use as a reporter gene. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al., (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Various promoters may be utilized in the context of the present invention to regulate the expression of a delivered FUS1 gene. Of particular interest are tissue-specific promoters or elements, which permit tissue selective or preferential expression of FUS1. Promoters that are active in cancer cells: for example, a promoter that is preferentially active in cancer cells is hTERT.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (Chandler et al., 1997).

One may include a polyadenylation signal in the expression construct to effect proper polyadenylation of the transcript. The nature of the polyadenylation signal is not believed to be crucial to the successful practice of the invention, and/or any such sequence may be employed. Specific embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

The vectors or constructs of the present invention may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Examples of selectable and screenable markers are well known to one of skill in the art.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781).

There are a number of ways in which FUS1 expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986).

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus vectors have been used in eukaryotic gene expression (Levrero et al., 1991; Gomez-Foix et al., 1992) and vaccine development (Grunhaus et al., 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975; Roizman and Sears, 1995). The expression of α genes, the first set of genes to be expressed after infection, is enhanced by the virion protein number 16, or α-transinducing factor (Post et al., 1981; Batterson and Roizman, 1983). The expression of β genes requires functional a gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

Adeno-Associated Virus Expression Vectors

Adeno-associated virus (AAV) has emerged as a potential alternative to the more commonly used retroviral and adenoviral vectors. While studies with retroviral and adenoviral mediated gene transfer raise concerns over potential oncogenic properties of the former, and immunogenic problems associated with the latter, AAV has not been associated with any such pathological indications.

In addition, AAV possesses several unique features that make it more desirable than the other vectors. Unlike retroviruses, AAV can infect non-dividing cells; wild-type AAV has been characterized by integration, in a site-specific manner, into chromosome 19 of human cells (Kotin and Berns, 1989; Kotin et al., 1990; Kotin et al., 1991; Samulski et al., 1991); and AAV also possesses anti-oncogenic properties (Ostrove et al., 1981; Berns and Giraud, 1996). Recombinant AAV genomes are constructed by molecularly cloning DNA sequences of interest between the AAV ITRs, eliminating the entire coding sequences of the wild-type AAV genome. The AAV vectors thus produced lack any of the coding sequences of wild-type AAV, yet retain the property of stable chromosomal integration and expression of the recombinant genes upon transduction both in vitro and in vivo (Berns, 1990; Berns and Bohensky, 1987; Kearns et al., 1996; Ponnazhagan et al., 1997a). Until recently, AAV was believed to infect almost all cell types, and even cross species barriers. However, it now has been determined that AAV infection is receptor-mediated (Ponnazhagan et al., 1996; Mizukami et al., 1996).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The sequence of AAV is provided in U.S. Pat. No. 5,252,479 (entire text of which is specifically incorporated herein by reference).

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and IL cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most, highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

A FUS1-encoding nucleic acid may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

In certain embodiments, a plasmid vector is contemplated for use in transferring FUS1 to cancer cells. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the host cell for the expression of PTEN.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Several non-viral methods for the transfer of expression constructs into mammalian cells also are contemplated by the present invention. In one embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest also may be transferred in a similar manner in vivo and express the gene product.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection. Of particular interest are the methods and compositions disclosed in PCT/US00/14350, incorporated by reference herein.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993). Receptor-mediated gene targeting vehicles generally consist of two components: a cell receptor-specific ligand and a DNA-binding agent. In other embodiments, the delivery vehicle may comprise a ligand and a liposome.

### Recombinant Polypeptide Expression

The ability to produce biologically active FUS1 polypeptide is an important aspect of the present invention. Development of mammalian cell culture for production of proteins has been greatly aided by the development in molecular biology of techniques for design and construction of vector systems highly efficient in mammalian cell cultures, a battery of useful selection markers, gene amplification schemes and a more comprehensive understanding of the biochemical and cellular mechanisms involved in procuring the final biologically-active molecule from the introduced vector. Such techniques and reagents are described elsewhere in this document.

The present invention can further take advantage of the biochemical and cellular capacities of cells to secrete FUS1, as well as of available bioreactor technology. Growing cells in a bioreactor allows for large scale production and secretion of complex, fully biologically-active FUS1 polypeptides into the growth media. Thus, engineered cells can act as factories for the production of large amounts of FUS1.

### Anchorage-Dependent Versus Non-Anchorage-Dependent Cultures

Animal and human cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. Large scale suspension culture based on microbial (bacterial and yeast) fermentation technology has clear advantages for the manufacturing of mammalian cell products. The processes are relatively simple to operate and straightforward to scale up. Homogeneous conditions can be provided in the reactor which allows for precise monitoring and control of temperature, dissolved oxygen, and pH, and ensure that representative samples of the culture can be taken.

However, suspension cultured cells cannot always be used in the production of biologicals. Suspension cultures are still considered to have tumorigenic potential and thus their use as substrates for production put limits on the use of the resulting products in human and veterinary applications (Petricciani, 1985; Larsson and Litwin, 1987). Viruses propagated in suspension cultures as opposed to anchorage-dependent cultures can sometimes cause rapid changes in viral markers, leading to reduced immunogenicity (Bahnemann, 1980). Finally, sometimes even recombinant cell lines can secrete considerably higher amounts of products when propagated as anchorage-dependent cultures as compared with the same cell line in suspension (Nilsson and Mosbach, 1987). For these reasons, different types of anchorage-dependent cells are used extensively in the production of different biological products.

### Modulators and Binding Compounds

The present invention provides methods for testing the functional effect of a test agent on a transgenic mammal, or on a cell derived from a transgenic mammal, with at least one disrupted FUS1 allele. In addition, the present invention provides methods for testing the functional effect of a test agent on FUS1 polypeptides and polynucleotides, and on cells expressing FUS1 polypeptides and polynucleotides. Such test agents can be any small compound, including polypeptides, polynucleotides, amino acids, nucleotides, carbohydrates, lipids, or any other organic or inorganic molecule. Alternatively, modulators can be genetically altered versions of a FUS1 gene. Typically, test compounds will be small chemical molecules and peptides. Essentially any chemical compound can be used as a potential modulator or binding compound in the assays of the invention, although often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Bucks, Switzerland) and the like.

To identify molecules capable of modulating FUS1, e.g., to identify compounds useful in the treatment or prevention of immune disorders or other FUS1-associated diseases and conditions, assays will often be performed to detect the effect of various compounds on FUS1 activity alone, or on FUS1 activity or expression in a cell. Such assays can involve the identification of compounds that interact with FUS1 proteins, either physically or genetically, and can thus rely on any of a number of standard methods to detect physical or genetic interactions between compounds. Such assays can also involve the identification of compounds that affect FUS1 expression, activity or other properties, such as its phosphorylation or ability to bind other proteins. Such assays can also involve the detection of FUS1 activity in a cell, either in vitro or in vivo.

### Assays for FUS1-Interacting Compounds

In certain embodiments, assays will be performed to identify molecules that physically or genetically interact with FUS1 proteins. Such molecules may represent molecules that normally interact with FUS1 or other molecules that are capable of interacting with FUS1 and that can potentially be used to modulate FUS1 activity in cells, or used as lead compounds to identify classes of molecules that can interact with and/or modulate FUS1. Such assays may represent physical binding assays, such as affinity chromatography, immunoprecipitation, two-hybrid screens, or other binding assays, or may represent genetic assays as described infra.

In any of the binding or functional assays described herein, in vivo or in vitro, any FUS1 protein, or any derivative, variation, homolog, or fragment of a FUS1 protein, can be used. In numerous embodiments, a fragment of a FUS1 protein is used. Such fragments can be used alone, in combination with other FUS1 fragments, or in combination with sequences from heterologous proteins, e.g., the fragments can be fused to a heterologous polypeptide, thereby forming a chimeric polypeptide.

### Assays for Physical Interactions

Compounds that interact with FUS1 proteins can be isolated based on an ability to specifically bind to a FUS1 protein or fragment thereof. In numerous embodiments, the FUS1 protein or protein fragment will be attached to a solid support. In one embodiment, affinity columns are made using the FUS1 polypeptide, and physically-interacting molecules are identified. It will be apparent to one of skill that chromatographic techniques can be performed at any scale and using equipment from many different manufacturers (e.g., Pharmacia Biotech). In addition, molecules that interact with FUS1 proteins in vivo can be identified by co-immunoprecipitation or other methods, i.e., immunoprecipitation FUS1 proteins using anti-FUS1 antibodies from a cell or cell extract, and identifying compounds, e.g., proteins, that are precipitated along with the FUS1 protein. Such methods are well known to those of skill in the art and are taught, e.g., in Ausubel et al, Sambrook et al., Harlow & Lane, all supra.

Two-hybrid screens can also be used to identify polypeptides that interact in vivo with a FUS1 polypeptide or a fragment thereof (Fields, et al., Nature, 340:245-246 (1989)). Such screens comprise two discrete, modular domains of a transcription factor protein, e.g., a DNA binding domain and a transcriptional activation domain, which are produced in a cell as two separate polypeptides, each of which also comprises one of two potentially binding polypeptides. If the two potentially binding polypeptides in fact interact in vivo, then the DNA binding and the transcriptional activating domain of the transcription factor are united, thereby producing expression of a target gene in the cell. The target gene typically encodes an easily detectable gene product, e.g., β-galactosidase, GFP, or luciferase, which can be detected using standard methods. In the present invention, a FUS1 polypeptide is fused to one of the two domains of the transcription factor, and the potential FUS1-binding polypeptides (e.g., encoded by a cDNA library) are fused to the other domain. Such methods are well known to those of skill in the art, and are taught, e.g., in Ausubel et al., supra.

### Assessing the Functional Effect of Test Agents on Mammals

In a number of embodiments, the effect of a test agent on a non-human mammal is assessed. For example, the effect of a known FUS1-modulating compound can be administered to an animal to assess the FUS1-independent effect of the compound on the animal. Such methods are useful, e.g., to detect possible side effects of a candidate FUS1-inhibiting drug. In addition, such methods can be used, e.g., to assess the effect of a suspected FUS1-modulating compound on FUS1 activity or expression in vise, or to screen for FUS1 modulating compounds.

The effects of the test compounds upon the function of any of the herein-described animals can also be measured by examining changes in any physiological process associated FUS1 activity. For example, one can measure a variety of effects such as changes in bone density, in lymphoid system development, in inflammation of tissues, as indicated by, e.g., pain, heat, redness, swelling, loss of function, dilatation of arterioles, capillaries and venules, with increased permeability and blood flow, exudation of fluids, including plasma proteins and leukocyte migration into the site of inflammation. In addition, any physiological effect can be detected, including any behavioral manifestation, any change in, e.g., temperature, blood pressure, viability, fertility, growth rate, organ function, etc. In addition, any assay or means of assessment described in the Examples, infra, can be used.

### Combinatorial Libraries

In one preferred embodiment, assessing the effects of a test agent on cells or animals, e.g., transgenic animals with at least one disrupted FUS1 allele, involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37:487-493 and Houghton, et al. (1991) Nature, 354:84-88). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs, et al. (1993) Proc. Nat. Acad. Sci. U.S.A., 90:6909-6913), vinylogous polypeptides (Hagihara, et al. (1992) J. Amer. Chem. Soc., 114:6568), nonpeptidal peptidomimetics with glucose scaffolding (Hirschmann, et al. (1992) J. Amer. Chem. Soc., 114:9217-9218), analogous organic syntheses of small compound libraries (Chen et al., J. Amer. Chem. Soc. 116:2661 (1994)), oligocarbiamates (Cho et al. (1993) Science, 261:1303), and/or peptidyl phosphonates (Campbell, et al. (1994) J. Org. Chem., 59:658), nucleic acid libraries (see Ausubel, Berger and Sambrook, all supra), peptide nucleic acid libraries (see, e.g., U.S. Pat. No. 5,539,083), antibody libraries (see, e.g., Vaughn, et al. (1996) Nature Biotechnology, 14(3):309-314 and PCT/US96/10287), carbohydrate libraries (see, e.g., Liang, et al. (1996) Science, 274:1520-1522 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainir, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

In embodiments involving isolated cells, high throughput assays may be used. In such high throughput assays, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

### Administration of FUS1 Modulators

To assess the effect of a test agent on an animal, or to treat or prevent a FUS1-associated condition in an animal, administration of a compound can be achieved by any of the routes normally used for introducing a modulator compound into ultimate contact with the tissue to be treated. The modulators are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17th ed. 1985)).

The FUS1 modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and nonaqueous solutions, isotonic sterile solutions, which can contain antioxidants, buffers, bacteriostats, and solutes that render the formulation isotonic, and aqueous and nonaqueous sterile suspensions that can include suspending agents, solubilizers, thickening agents, stabilizers, and preservatives. In the practice of this invention, compositions can be administered, for example, orally, nasally, topically, intravenously, intraperitoneally, intravesically or intrathecally. The formulations of compounds can be presented in unit-dose or multi-dose scaled containers, such as ampules and vials. Solutions and suspensions can be prepared from sterile powders, granules, and tablets of the kind previously described. The modulators can also be administered as part a of prepared food or drug.

The dose administered to a patient, in the context of the present invention is often varied to assess the effect of various concentrations of a compound on a transgenic animal. The dose will also be determined by, e.g., the body weight or surface area of the area to be exposed to the compound. In general, the dose equivalent of a modulator is from about 1 ng/kg to 10 mg/kg for a typical subject. Administration can be accomplished via single or divided doses.

### Generation of Targeting Construct

The targeting construct of the present invention may be produced using standard methods known in the art. (see, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chain reaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

The targeting construct of the present invention typically comprises a first sequence homologous to a portion or region of the FUS1 gene and a second sequence homologous to a second portion or region of the FUS1 gene. The targeting construct further comprises a positive selection marker, which is preferably positioned in between the first and the second DNA sequence that are homologous to a portion or region of the target DNA sequence. The positive selection marker may be operatively linked to a promoter and a polyadenylation signal.

Other regulatory sequences known in the art may be incorporated into the targeting construct to disrupt or control expression of a particular gene in a specific cell type. In addition, the targeting construct may also include a sequence coding for a screening marker, for example, green fluorescent protein (GFP), or another modified fluorescent protein.

Although the size of the homologous sequence is not critical and can range from as few as 50 base pairs to as many as 100 kb, preferably each fragment is greater than about 1 kb in length, more preferably between about 1 and about 10 kb, and even more preferably between about 1 and about 5 kb. One of skill in the art will recognize that although larger fragments may increase the number of homologous recombination events in ES cells, larger fragments will also be more difficult to clone.

In a preferred embodiment of the present invention, the targeting construct is prepared directly from a plasmid genomic library using the methods described in pending U.S. patent application Ser. No. 08/971,310, filed Nov. 17, 1997, the disclosure of which is incorporated herein in its entirety. Generally, a sequence of interest is identified and isolated from a plasmid library in a single step using, for example, long-range PCR. Following isolation of this sequence, a second polynucleotide that will disrupt the target sequence can be readily inserted between two regions encoding the sequence of interest. In accordance with this aspect, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid.

In another embodiment, the targeting construct is designed in accordance with the regulated positive selection method described in U.S. patent application Ser. No. 60/232,957, filed Sep. 15, 2000, the disclosure of which is incorporated herein in its entirety. The targeting construct is designed to include a PGK-neo fusion gene having two lacO sites, positioned in the PGK promoter and an NLS-lacI gene comprising a lac repressor fused to sequences encoding the NLS from the SV40 T antigen.

In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal. (see, e.g., U.S. Pat. No. 5,464,764; U.S. Pat. No. 5,487,992; U.S. Pat. No. 5,627,059; and U.S. Pat. No. 5,631,153).

### Generation of Cells and Confirmation of Homologous Recombination Events

Once an appropriate targeting construct has been prepared, the targeting construct may be introduced into an appropriate host cell using any method known in the art. Various techniques may be employed in the present invention, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyornithine, etc., or the like (see, e.g., U.S. Pat. No. 4,873,191; Van der Putten, et al., 1985, Proc. Natl. Acad. Sci., USA 82:6148-6152; Thompson, et al., 1989, Cell 56:313-321; Lo, 1983, Mol. Cell. Biol. 3:1803-1814; Lavitrano, et al., 1989, Cell, 57:717-723). Various techniques for transforming mammalian cells are known in the art. (see, e.g., Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

In a preferred aspect of the present invention, the targeting construct is introduced into host cells by electroporation. In this process, electrical impulses of high field strength reversibly permeabilize biomembranes allowing the introduction of the construct. The pores created during electroporation permit the uptake of macromolecules such as DNA. (see, e.g., Potter, H., et al., 1984, Proc. Nat'l. Acad. Sci. U.S.A. 81:7161-7165).

Any cell type capable of homologous recombination may be used in the practice of the present invention. Examples of such target cells include cells derived from vertebrates including mammals such as humans, bovine species, ovine species, murine species, simian species, and ether eukaryote organisms such as filamentous fungi, and higher multicellular organisms such as plants.

Preferred cell types include embryonic stem (ES) cells, which are typically obtained from pre-implantation embryos cultured in vitro. (see, e.g., Evans, M. J., et al., 1981, Nature 292:154-156; Bradley, M. O., et al., 1984, Nature 309:255-258; Gossler et al., 1986, Proc. Natl. Acad. Sci. USA 83:9065-9069; and Robertson, et al., 1986, Nature 322:445-448). The ES cells are cultured and prepared for introduction of the targeting construct using methods well known to the skilled artisan. (see, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA, 88:10730; Dorin et al., 1992, Transgenic Res. 1:101; and Veis et al., 1993, Cell 75:229). The ES cells that will be inserted with the targeting construct are derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development. Thus, any ES cell line having this capability is suitable for use in the practice of the present invention.

The present invention may also be used to knockout genes in other cell types, such as stem cells. By way of example, stem cells may be myeloid, lymphoid, or neural progenitor and precursor cells. These cells comprising a disruption or knockout of a gene may be particularly useful in the study of FUS1 gene function in individual developmental pathways. Stem cells may be derived from any vertebrate species, such as mouse, rat, dog, cat, pig, rabbit, human, non-human primates and the like.

After the targeting construct has been introduced into cells, the cells where successful gene targeting has occurred are identified. Insertion of the targeting construct into the targeted gene is typically detected by identifying cells for expression of the marker gene. In a preferred embodiment, the cells transformed with the targeting construct of the present invention are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce.

If a regulated positive selection method is used in identifying homologous recombination events, the targeting construct is designed so that the expression of the selectable marker gene is regulated in a manner such that expression is inhibited following random integration but is permitted (derepressed) following homologous recombination. More particularly, the transfected cells are screened for expression of the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). (see, e.g., Mansour et al., Nature 336:348-352: (1988); Capecchi, Science 244:1288-1292, (1989); Capecchi, Trends in Genet. 5:70-76 (1989)).

Successful recombination may be identified by analyzing the DNA of the selected cells to confirm homologous recombination. Various techniques known in the art, such as PCR and/or Southern analysis may be used to confirm homologous recombination events.

Homologous recombination may also be used to disrupt genes in stem cells, and other cell types, which are not totipotent embryonic stem cells. By way of example, stem cells may be myeloid, lymphoid, or neural progenitor and precursor cells. Such transgenic cells may be particularly useful in the study of FUS1 gene function in individual developmental pathways. Stem cells may be derived from any vertebrate species, such as mouse, rat, dog, cat, pig, rabbit, human, non-human primates and the like.

In cells that are not totipotent it may be desirable to knock out both copies of the target using methods that are known in the art. For example, cells comprising homologous recombination at a target locus that have been selected for expression of a positive selection marker (e.g., Neo^{r}) and screened for non-random integration, can be further selected for multiple copies of the selectable marker gene by exposure to elevated levels of the selective agent (e.g., G418). The cells are then analyzed for homozygosity at the target locus. Alternatively, a second construct can be generated with a different positive selection marker inserted between the two homologous sequences. The two constructs can be introduced into the cell either sequentially or simultaneously, followed by appropriate selection for each of the positive marker genes. The final cell is screened for homologous recombination of both alleles of the target.

### Production of Transgenic Animals

Selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected ES cells can be allowed to aggregate with dissociated mouse embryo cells to form the aggregation chimera. A chimeric embryo can then be implanted into a suitable pseudopregnant female foster animal and the embryo brought to term. Chimeric progeny harbouring the homologously recombined DNA in their germ cells can be used to breed animals in which all cells of the animal contain the homologously recombined DNA. In one embodiment, chimeric progeny mice are used to generate a mouse with a heterozygous disruption in the FUS1 gene. Heterozygous transgenic mice can then be mated. It is well know in the art that typically ¼ of the offspring of such matings will have a homozygous disruption in the FUS1 gene.

The heterozygous and homozygous transgenic mice can then be compared to normal, wild type mice to determine whether disruption of the FUS1 gene causes phenotypic changes, especially pathological changes. For example, heterozygous and homozygous mice may be evaluated for phenotypic changes by physical examination, necropsy, histology, clinical chemistry, complete blood count, body weight, organ weights, and cytological evaluation of bone marrow.

In one embodiment, the phenotype (or phenotypic change) associated with a disruption in the FUS1 gene is placed into or stored in a database. Preferably, the database includes: (i) genotypic data (e.g., identification of the disrupted gene) and (ii) phenotypic data (e.g., phenotype(s) resulting from the gene disruption) associated with the genotypic data. The database is preferably electronic. In addition, the database is preferably combined with a search tool so that the database is searchable.

### Conditional Transgenic Animals

The present invention further contemplates conditional transgenic or knockout animals, such as those produced using recombination methods. Bacteriophage P1 Cre recombinase and flp recombinase from yeast plasmids are two non-limiting examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present invention. Such recombinases include the Int recombinase of bacteriophage .lambda. (with or without X is) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the .beta.-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec. Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al., (Mol. Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxP site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. When the constructs described herein are used to disrupt FUS1 genes, a fusion transcript can be produced when insertion of the positive selection marker occurs downstream (3′) of the translation initiation site of the FUS1 gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs that allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, promoters. Vectors incorporating such promoters would only express recombinase activity in cells that express the necessary transcription factors.

### Models for Disease

The cell- and animal-based systems described herein can be utilized as models for diseases. Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. Such assays may be utilized as part of screening strategies designed to identify agents, such as compounds that are capable of ameliorating disease symptoms. Thus, the animal- and cell-based models may be used to identify drugs, pharmaceuticals, therapies and interventions that may be effective in treating disease.

Cell-based systems may be used to identify compounds that may act to ameliorate disease symptoms. For example, such cell systems may be exposed to a compound suspected of exhibiting an ability to ameliorate disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild type, non-disease phenotype.

In addition, animal-based disease systems, such as those described herein, may be used to identify compounds capable of ameliorating disease symptoms. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the disease or phenotype. Neonatal, juvenile, and adult animals can also be exposed.

More particularly, using the animal models of the invention, specifically, transgenic mice, methods of identifying agents, including compounds are provided, preferably, on the basis of the ability to affect at least one phenotype associated with a disruption in a FUS1 gene. In one embodiment, the present invention provides a method of identifying agents having an effect on FUS1 expression or function. The method includes measuring a physiological response of the animal, for example, to the agent, and comparing the physiological response of such animal to a control animal, wherein the physiological response of the animal comprising a disruption in a FUS1 as compared to the control animal indicates the specificity of the agent. A “physiological response” is any biological or physical parameter of an animal that can be measured. Molecular assays (e.g., gene transcription, protein production and degradation rates), physical parameters (e.g., exercise physiology tests, measurement of various parameters of respiration, measurement of heart rate or blood pressure, measurement of bleeding time, a PTT.T, or TT), and cellular assays (e.g., immunohistochemical assays of cell surface markers, or the ability of cells to aggregate or proliferate) can be used to assess a physiological response.

The transgenic animals and cells of the present invention may be utilized as models for diseases, disorders, or conditions associated with phenotypes relating to a disruption in a FUS1.

### FUS1 Gene Products

The present invention further contemplates use of the FUS1 gene sequence to produce FUS1 gene products. FUS1 gene products may include proteins that represent functionally equivalent gene products. Such an equivalent gene product may contain deletions, additions or substitutions of amino acid residues within the amino acid sequence encoded by the gene sequences described herein, but which result in a silent change, thus producing a functionally equivalent FUS1 gene product Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a protein capable of exhibiting a substantially similar in vivo activity as the endogenous gene products encoded by the FUS1 gene sequences. Alternatively, when utilized as part of an assay, “functionally equivalent” may refer to peptides capable of interacting with other cellular or extracellular molecules in a manner substantially similar to the way in which the corresponding portion of the endogenous gene product would.

Other protein products useful according to the methods of the invention are peptides derived from or based on the FUS1 gene produced by recombinant or synthetic means (derived peptides).

FUS1 gene products may be produced by recombinant DNA technology using techniques well known in the art. Thus, methods for preparing the gene polypeptides and peptides of the invention by expressing nucleic acid encoding gene sequences are described herein. Methods that are well known to those skilled in the art can be used to construct expression vectors containing gene protein coding sequences and appropriate transcriptional/translational control signals. These methods include, for example, in vitro recombinant DNA techniques, synthetic techniques and in vivo recombination/genetic recombination (see, e.g., Sambrook, et al., 1989, supra, and Ausubel, et al., 1989, supra). Alternatively, RNA capable of encoding gene protein sequences may be chemically synthesized using, for example, automated synthesizers (see, e.g. Oligonucleotide Synthesis: A Practical Approach, Gait, M. J. ed., IRL Press, Oxford (1984)).

A variety of host-expression vector systems may be utilized to express the gene coding sequences of the invention. Such host-expression systems represent vehicles by which the coding sequences of interest may be produced and subsequently purified, but also represent cells that may, when transformed or transfected with the appropriate nucleotide coding sequences, exhibit the gene protein of the invention in situ. These include but are not limited to microorganisms such as bacteria (e.g., E. coli, B. subtilis) transformed with recombinant bacteriophage DNA, plasmid DNA or cosmid DNA expression vectors containing gene protein coding sequences; yeast (e.g. Saccharomyces, Pichia) transformed with recombinant yeast expression vectors containing the gene protein coding sequences; insect cell systems infected with recombinant virus expression vectors (e.g., baculovirus) containing the gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing gene protein coding sequences; or mammalian cell systems (e.g. COS, CHO, BHK, 293, 3T3) harboring recombinant expression constructs containing promoters derived from the genome of mammalian cells (e.g., metallothionine promoter) or from mammalian viruses (e.g., the adenovirus late promoter; the vaccinia virus 7.5 K promoter).

In bacterial systems, a number of expression vectors may be advantageously selected depending upon the use intended for the gene protein being expressed. For example, when a large quantity of such a protein is to be produced, for the generation of antibodies or to screen peptide libraries, for example, vectors that direct the expression of high levels of fusion protein products that are readily purified may be desirable. Such vectors include, but are not limited, to the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned FUS1 gene protein can be released from the GST moiety.

In an insect system, Autographa californica nuclear polyhedrosis virus (AcNPV) is used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith, et al., J. Virol. 46:584-93 (1983); U.S. Pat. No. 4,745,051).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing gene protein in infected hosts. (e.g., see Logan et al., Proc. Natl. Acad. Sci. USA, 81:3655-59 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter, et al., Methods in Enzymol., 153:516-44 (1987)).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

For long-term, high-yield production of recombinant proteins, stable expression is preferred. For example, cell lines that stably express the gene protein may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells that stably integrate the plasmid into their chromosomes and grow, to form foci, which in turn can be cloned and expanded into cell lines. This method may advantageously be used to engineer cell lines that express the gene protein. Such engineered cell lines may be particularly useful in screening and evaluation of compounds that affect the endogenous activity of the gene protein.

In a preferred embodiment, timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one in embodiment, a Tet inducible gene expression system is utilized. (Gossen et al., Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon—the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels. Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include but are not limited to polyclonal, monoclonal, chimeric, single chain, Fab fragments and fragments produced by a Fab expression library.

### Production of Antibodies

Described herein are methods for the production of antibodies capable of specifically recognizing one or more epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a FUS1 gene in a biological sample, or, alternatively, as a method for the inhibition of abnormal FUS1 gene activity. Thus, such antibodies may be utilized as part of disease treatment methods, and/or may be used as part of diagnostic techniques whereby patients may be tested for abnormal levels of FUS1 gene proteins, or for the presence of abnormal forms of such proteins.

For the production of antibodies, various host animals may be immunized by injection with the FUS1 gene, its expression product or a portion thereof. Such host animals may include but are not limited to rabbits, mice, rats, goats and chickens, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as FUS1 gene product, or an antigenic functional derivative thereof. For the production of polyclonal antibodies, host animals such as those described above, may be immunized by injection with gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

### Screening Methods

The present invention may be employed in a process for screening for agents such as agonists, i.e., agents that bind to and activate FUS1 polypeptides. Thus, polypeptides of the invention may also be used to assess the binding of small molecule substrates and ligands in, for example, cells, cell-free preparations, chemical libraries, and natural product mixtures as known in the art. Any methods routinely used to identify and screen for agents that can modulate receptors may be used in accordance with the present invention.

The present invention provides methods for identifying and screening for agents that modulate FUS1 expression or function. More particularly, cells that contain and express FUS1 gene sequences may be used to screen for therapeutic agents. Such cells may include non-recombinant monocyte cell lines, such as U937 (ATCC# CRL-1593), THP-1 (ATCC# TIB-202), and P388D1 (ATCC# TIB-63); endothelial cells such as HLVEC's and bovine aortic endothelial cells (BAEC's); as well as generic mammalian cell lines such as HeLa cells and COS cells, e.g., COS-7 (ATCC# CRL-1651). Further, such cells may include recombinant, transgenic cell lines. For example, the transgenic mice of the invention may be used to generate cell lines, containing one or more cell types involved in a disease, that can be used as cell culture models for that disorder. While cells, tissues, and primary'cultures derived from the disease transgenic animals of the invention may be utilized, the generation of continuous cell lines is preferred. For examples of techniques that may be used to derive a continuous cell line from the transgenic animals, see Small, et al., Mol. Cell. Biol., 5:642-48 (1985).

FUS1 gene sequences may be introduced into, and overexpressed in, the genome of the cell of interest. In order to overexpress a FUS1 gene sequence, the coding portion of the FUS1 gene sequence may be ligated to a regulatory sequence that is capable of driving gene expression in the cell type of interest. Such regulatory regions will be well known to those of skill in the art, and may be utilized in the absence of undue experimentation. FUS1 gene sequences may also be disrupted or underexpressed. Cells having FUS1 gene disruptions or underexpressed FUS1 gene sequences may be used, for example, to screen for agents capable of affecting alternative pathways that compensate for any loss of function attributable to the disruption or underexpression.

In vitro systems may be designed to identify compounds capable of binding the FUS1 gene products. Such compounds may include, but are not limited to, peptides made of D- and/or L-configuration amino acids (m, for example, the form of random peptide libraries; (see e.g., Lam, et al., Nature, 354:82-4 (1991)), phosphopeptides (m, for example, the form of random or partially degenerate, directed phosphopeptide libraries; see, e.g., Songyang, et al., Cell, 72:767-78 (1993)), antibodies, and small organic or inorganic molecules. Compounds identified may be useful, for example, in modulating the activity of FUS1 gene proteins, preferably mutant FUS1 gene proteins; elaborating the biological function of the FUS1 gene protein; or screening for compounds that disrupt normal FUS1 gene interactions or themselves disrupt such interactions.

The principle of the assays used to identify compounds that bind to the FUS1 gene protein involves preparing a reaction mixture of the FUS1 gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring the FUS1 gene protein or the test substance onto a solid phase and detecting target protein/test substance complexes anchored on the solid phase at the end of the reaction. In one embodiment of such a method, the FUS1 gene protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for FUS1 gene product or the test compound to anchor any complexes formed in solution, and a labeled antibody specific for the other component of the possible complex to detect anchored complexes.

Compounds that are shown to bind to a particular FUS1 gene product through one of the methods described above can be further tested for their ability to elicit a biochemical response from the FUS1 gene protein. Agonists, antagonists and/or inhibitors of the expression product can be identified utilizing assays well known in the art.

### Antisense, Ribozymes, and Antibodies

Other agents that may be used as therapeutics include the FUS1 gene, its expression product(s) and functional fragments thereof. Additionally, agents that reduce or inhibit mutant FUS1 gene activity may be used to ameliorate disease symptoms or to study disease symptoms. Such agents include antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the FUS1 gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to the FUS1 gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding FUS1 gene proteins.

Specific ribozyme cleavage sites within any potential RNA target are initially identified by scanning the molecule of interest for ribozyme cleavage sites that include the following sequences, GUA, GUU and GUC. Once identified, short RNA sequences of between 15 and 20 ribonucleotides corresponding to the region of the FUS1 gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant FUS1 gene alleles. To ensure that substantially normal levels of FUS1 gene activity are maintained, nucleic acid molecules that encode and express FUS1 gene polypeptides exhibiting normal activity may be introduced into cells that do not contain sequences susceptible to whatever antisense, ribozyme, or triple helix treatments are being utilized. Alternatively, it may be preferable to coadminister normal FUS1 gene protein into the cell or tissue in order to maintain the requisite level of cellular or tissue FUS1 gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for FUS1 gene protein, and in particular, mutant gene protein, and interfere with its activity may be used to inhibit mutant FUS1 gene function. Such antibodies may be generated against the proteins themselves or against peptides corresponding to portions of the proteins using standard techniques known in the art and as also described herein. Such antibodies include but are not limited to polyclonal, monoclonal, Fab fragments, single chain antibodies, chimeric antibodies, etc.

In instances where the FUS1 gene protein is intracellular and whole antibodies are used, internalizing antibodies may be preferred. However, lipofectin liposomes may be used to deliver the antibody or a fragment of the Fab region that binds to the FUS1 gene epitope into cells. Where fragments of the antibody are used, the smallest inhibitory fragment that binds to the target or expanded target protein's binding domain is preferred. For example, peptides having an amino acid sequence corresponding to the domain of the variable region of the antibody that binds to the FUS1 gene protein may be used. Such peptides may be synthesized chemically or produced via recombinant DNA technology using methods well known in the art (see, e.g., Creighton, Proteins: Structures and Molecular Principles (1984) W. H. Freeman, New York 1983, supra; and Sambrook, et al., 1989, supra). Alternatively, single chain neutralizing antibodies that bind to intracellular FUS1 gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

RNA sequences encoding FUS1 gene protein may be directly administered to a patient exhibiting disease symptoms, at a concentration sufficient to produce a level of FUS1 gene protein such that disease symptoms are ameliorated. Patients may be treated by gene replacement therapy. One or more copies of a normal FUS1 gene, or a portion of the gene that directs the production of a normal FUS1 gene protein with FUS1 gene function, may be inserted into cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal FUS1 gene sequences into human cells.

Cells, preferably, autologous cells, containing normal FUS1 gene expressing gene sequences may then be introduced or reintroduced into the patient at positions that allow for the amelioration of disease symptoms.

### Pharmaceutical Compositions, Effective Dosages, and Routes of Administration

The identified compounds that inhibit target mutant gene expression, synthesis and/or activity can be administered to a patient at therapeutically effective doses to treat or ameliorate the disease. A therapeutically effective dose refers to that amount of the compound sufficient to result in amelioration of symptoms of the disease.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral, topical, subcutaneous, intraperitoneal, intravenous, intrapleural, intraocular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

Pharmaceutical compositions may also include various buffers (e.g., Tris, acetate, phosphate), solubilizers (e.g., Tween, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as tween-20, tween-80, NP-40 or Triton X-100. EBP may also be incorporated into particulate preparations of polymeric compounds for controlled delivery to a patient over an extended period of time. A more extensive survey of components in pharmaceutical compositions is found in Remington's Pharmaceutical Sciences, 18th ed., A. R. Gennaro, ed., Mack Publishing, Easton, Pa. (1990).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### Diagnostics

A variety of methods may be employed to diagnose disease conditions associated with the FUS1 gene. Specifically, reagents may be used, for example, for the detection of the presence of FUS1 gene mutations, or the detection of either over or under expression of FUS1 gene mRNA.

According to the diagnostic and prognostic method of the present invention, alteration of the wild-type FUS1 gene locus is detected. In addition, the method can be performed by detecting the wild-type FUS1 gene locus and confirming the lack of a predisposition or neoplasia. “Alteration of a wild-type gene” encompasses all forms of mutations including deletions, insertions and point mutations in the coding and noncoding regions. Deletions may be of the entire gene or only a portion of the gene. Point mutations may result in stop codons, frameshift mutations or amino acid substitutions. Somatic mutations are those that occur only in certain tissues, e.g., in tumor tissue, and are not inherited in the germline. Germline mutations can be found in any of a body's tissues and are inherited. If only a single allele is somatically mutated, an early neoplastic state may be indicated. However, if both alleles are mutated, then a late neoplastic state may be indicated. The finding of gene mutations thus provides both diagnostic and prognostic information. A FUS1 gene allele that is not deleted (e.g., that found on the sister chromosome to a chromosome carrying a FUS1 gene deletion) can be screened for other mutations, such as insertions, small deletions, and point mutations. Mutations found in diseased tissues may be linked to decreased expression of the FUS1 gene product. However, mutations leading to non-functional gene products may also be linked to a diseased state. Point mutational events may occur in regulatory regions, such as in the promoter of the gene, leading to loss or diminution of expression of the mRNA. Point mutations may also abolish proper RNA processing, leading to loss of expression of the FUS1 gene product, or a decrease in mRNA stability or translation efficiency.

One test available for detecting mutations in a candidate locus is to directly compare genomic target sequences from cancer patients with those from a control population. Alternatively, one could sequence messenger RNA after amplification, e.g., by PCR, thereby eliminating the necessity of determining the exon structure of the candidate gene. Mutations from cancer patients falling outside the coding region of the FUS1 gene can be detected by examining the non-coding regions, such as introns and regulatory sequences near or within the FUS1 gene. An early indication that mutations in noncoding regions are important may come from Northern blot experiments that reveal messenger RNA molecules of abnormal size or abundance in cancer patients as compared to control individuals.

The methods described herein may be performed, for example, by utilizing prepackaged diagnostic kits comprising at least one specific gene nucleic acid or anti-gene antibody reagent described herein, which may be conveniently used, e.g., in clinical settings, to diagnose patients exhibiting disease symptoms or at risk for developing disease.

Any cell type or tissue, preferably brain, cortex, subcortical region, cerebellum, brainstem, eye, heart, lung, liver, pancreas, kidneys, skin, gallbladder, urinary bladder, pituitary gland, adrenal gland, salivary gland, tongue, stomach, large intestine, cecum, testis, epididymis, seminal vesicle, coagulating gland, prostate gland, ovary and uterus in which the gene is expressed may be utilized in the diagnostics described below.

DNA or RNA from the cell type or tissue to be analyzed may easily be isolated using procedures that are well known to those in the art. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, PCR In Situ Hybridization: Protocols and Applications, Raven Press, N.Y. (1992)).

### EXAMPLES

It should be appreciated that the invention should not be construed to be limited to the examples which are now described; rather, the invention should be construed to include any and all applications provided herein and all equivalent variations within the skill of the ordinary artisan.

### Example 1

Bioinformatics data on the Fus1 gene and protein. The Fus1 gene residing in the 3p21.3 chromosome region may function as a classical or haploinsufficient tumor suppressor. Fus1 is highly conserved and orthologs are present in rodent, chicken, fish, and worm genomes but not in the fly. The intron-exon structure of Fus1 is also conserved. Two intronless pseudogenes are present on chromosomes X and Y. The gene products are small, basic, (in human, 110 amino acids, pI 9.69) soluble, globular proteins mostly located in mitochondria but could be secreted by a non-classical and leaderless mechanism (http://www.cbs.dtu.dk/services/). ProDom identifies a single domain of unknown function, while PFAM and SMART do not indicate the presence of known domains. The proteins undergo extensive posttranslational modifications of which N-myristoylation of the NH2-terminal glycine residue was experimentally confirmed for human protein (12). The bioinformatics analysis suggests that these proteins may function as intracellular regulatory peptides in separate cellular compartments and as secreted signaling molecules. Fus1 is highly expressed in mouse development and in mouse embryonic stem cells (ESC) as expected for a cancer-causing gene ((http://lgsun.grc.nia.nih.gov/cDNA/cDNA.html). The tumor suppressor function of Fus1 was confirmed experimentally (3).

### Fus1 Expression in Various Non-Immune and Immune Tissues and Cells

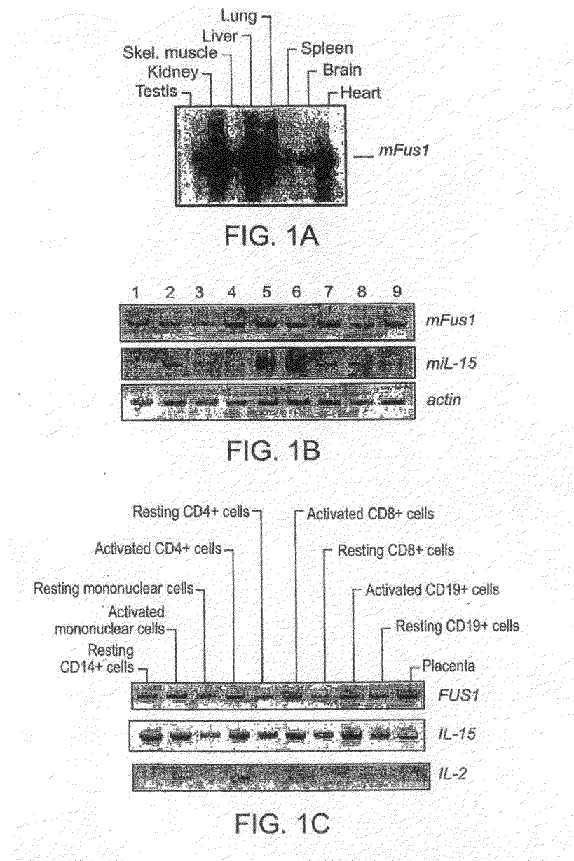

Northern blot analysis of wild type (WT) mouse tissues showed ubiquitous expression of Fus1 mRNA with the highest levels in kidney, liver, heart, and lungs (FIG. 1A). This distribution is similar to the distribution previously reported in human (1). Due to the immunological phenotype observed in Fus1-deficient mice (see below), Fus1 expression in lymphoid tissues was examined in detail. PCR analysis on Clontech cDNA MTCII panel revealed that Fus1 mRNA is present in murine thymus, lymph node, and bone marrow (FIG. 1B, lanes 7 to 9). Fus1 expression in spleen was demonstrated by Northern blot (FIG. 1A). Surprisingly, a high expression level of Fus1, similar to that in liver, was detected in the eye (FIG. 1B, lanes 1 and 4). To further delineate lymphoid cells that express Fus1, PCR analysis was performed on cDNAs isolated from human resting and activated T and B cells (“Clontech”, Human blood fractions MTC panel). Fus1 expression was detected in all blood peripheral immune cell subpopulations examined (FIG. 1C). In particular, Fus1 mRNA was detected in both T and B cells with a significantly increased level in activated T cells. A similar pattern was obtained for IL-15 expression in all tested subpopulations except B cells. The level of IL-15 expression in activated B cells was significantly elevated as compared to resting B cells, while Fus1 showed uniform expression in these two lymphocyte subpopulations (FIG. 1C). IL-2 (expressed only in activated T cells) was used as a control for cell purity (FIG. 1C).

The FUS1 Protein is Associated Predominantly with Mitochondria

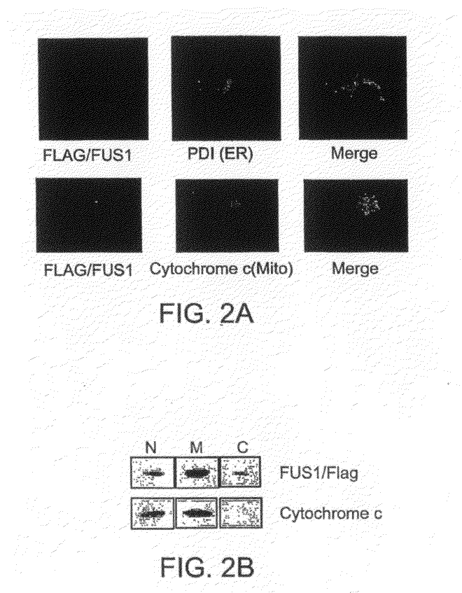

Fluorescent antibody (Ab) staining of 293T cells over-expressing the FUS1/FLAG protein with anti-FLAG Ab revealed a punctate cytoplasmic pattern (FIG. 2A). To identify FUS1-associated organelles, cells were co-stained with an anti-FLAG Ab, specific for the FUS1 protein, and Abs specific either to endoplasmic reticulum (ER), mitochondria, or Golgi apparatus. The anti-FLAG/FUS1 immunostaining pattern displayed no similarity with the one obtained with the anti-Golgi staining (data not shown). The anti-cytochrome c staining, that detects an inter-membrane resident of mitochondria, displayed a dotted network pattern in the cytoplasm that overlapped with the anti-FLAG/FUS1 staining in most areas (FIG. 2A). As expected, the anti-ER-resident protein disulfide isomerase (PDI) staining displayed a fine reticular pattern adjacent to the nucleus (FIG. 2A). Although there were some overlapping areas, the ER staining pattern in general was distinct from that of the FUS1 protein indicating that FUS1 is not a resident ER protein (FIG. 2A).

Data on fractionation of 293T cells over-expressing the FLAG/FUS1 protein confirmed the immunofluorescence data (FIG. 2B). Western blot analysis of the different cell fractions revealed an intense band corresponding in size to the FUS1 protein in the mitochondrial fraction (M), while only a small amount was found in the cytoplasmic fraction (C). Also detected was a residual level in the nuclear fraction (N) that was probably due to contamination from the cytoplasmic fraction since Cytochrome c, a mitochondrial resident used as a control for the fraction purity, was also detected in the nucleus.

Taken together, these results indicate that FUS1 is a mitochondrial resident, capable of shuttling to the ER. No reliable data on a FUS1 nuclear localization have been obtained.

### Generation of Mice Lacking the Fus1 Gene

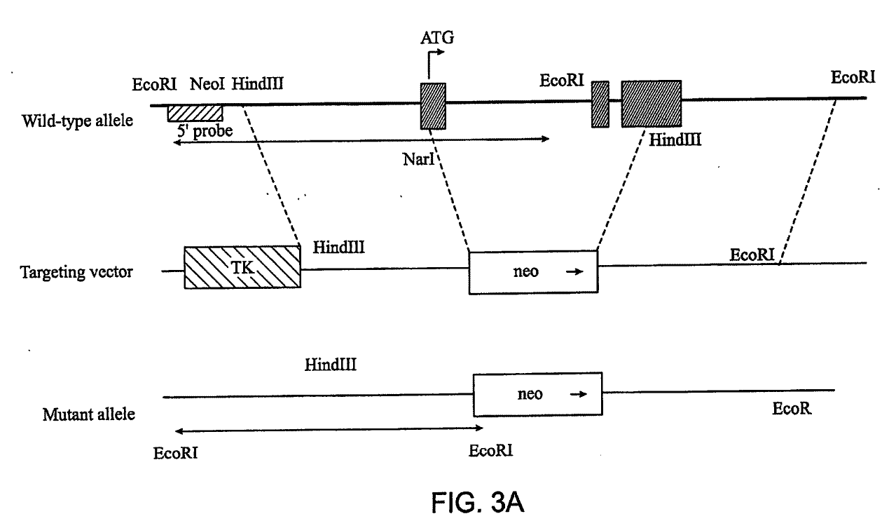

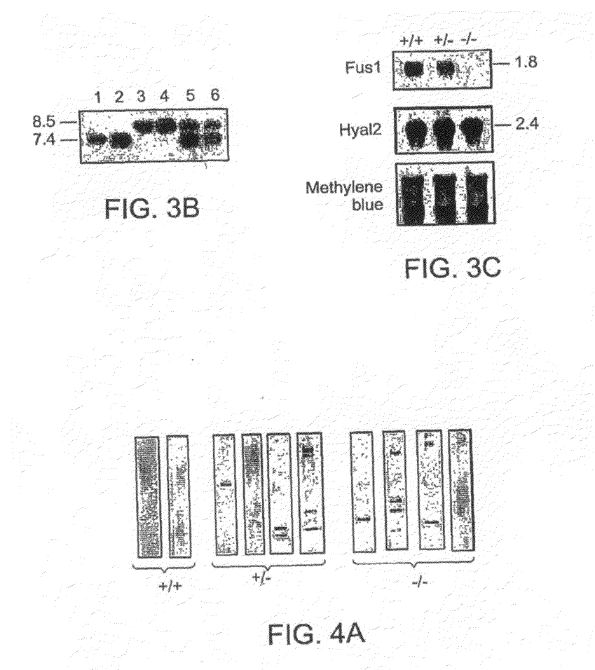

Fus1-deficient mice were generated through homologous recombination in cultured embryonic stem cells. The targeting vector replaced part of the first Fus1 exon, including the translation initiation codon, the entire second exon and a part of the third exon, leaving in the recombinant allele, the 3′-noncoding Fus1 region fused with a neo gene cassette (FIG. 3A). This replacement was confirmed by Southern blot analysis using a probe external to the 5′ end of the construct (FIG. 3B). Successfully targeted embryonic stem cells were injected into C57BL/6J blastocysts, and chimeric males were bred with C57BL/6J females to produce F₁ hybrid (129/Sv×C57BL/6) heterozygotes. The F₁ hybrid mice were inbred to generate F₂ and F₃ hybrid progeny. Northern blotting on brain mRNA confirmed that the Fus1 mRNA level was reduced in Fus1^{+/-} mice and undetectable in Fus1^{-/-} mice (FIG. 3C). Re-probing of the blot revealed that expression of the bordering Hyal2 gene was unaffected (FIG. 3C). Young Fus1^{+/-} and Fus1^{-/-} mice are viable, fertile, and externally/internally undistinguishable from WT mice. The only difference found between WT and knockout mice was a ˜20% reduction in the Fus1^{-/-} body weight by the age of 6 months measured in sex-matched cohorts (data not shown).

### Mice Lacking the FUS1 Protein are Predisposed to Premature Death

Although histological examination of randomly chosen young Fus1^{+/-} and Fus1^{-/-} mice revealed no evident abnormalities, ˜11% of Fus1^{+/-} (15/132) and ˜11% of Fus1^{-/-} (7/64) mice died between 2 and 14 months. No premature deaths were reported in the WT cohort (0/52). To investigate the cause of early death, necropsy and histopathological analysis of tissues from 2 to 14 month old mice that died or were sacrificed due to clinical manifestations (dehydration, fur ruffling, lethargy, difficulties in breathing) was performed. The abnormalities revealed in this analysis are listed in Table I. Observed were signs of systemic infection (thymus atrophy, spleen depletion, bone marrow congestion) in some animals. However, these signs are not likely to be related to the increased mortality. More acute pathological signs were also recognized, such as fibrinoid necrotizing arteritis in multiple organs, severe GN with tubular casts, severe nephropathy and anemia, that may be directly responsible for the premature death. The WT age-matched cohort was completely free of all these abnormalities. Also detected were tumors in a few of the animals that died prematurely (Table I).

### Spontaneous Development of Systemic Vasculitis in Fus1^{+/-} and Fus1^{-/-} Mice

The triad of symptoms characteristic for an autoimmune disorder, i.e. arteritis, severe kidney abnormalities and anemia, was seen in a few Fus1^{+/-} and Fus1^{-/-} mice that died prematurely (Table I). WT mice never showed any histological evidence of arteritis, severe GN or anemia at the same age or even at older ages (observations, 13). To compare the frequencies of these symptoms in aging mice, the group of 27 WT, 39 Fus1^{+/-} and 25 Fus1^{-/-} was analyzed that included mice that either died or were sacrificed upon reaching 2 years old. Among these, 23% of Fus1^{+/-} and 20% of Fus1^{-/-} mice developed arteritis in either single or multiple organs. Since Fus1^{+/-} and Fus1^{-/-} mice demonstrated similar phenotypic changes, both groups of mice were pooled for statistical analysis. A Fisher's exact test was used to investigate the existence of a potential relation between Fus1 disruption and the presence of arteritis. The analysis revealed a statistically significant association between these two parameters (p=0.0323). The association of Fus1^{-/-} with the development of moderate or severe kidney GN and/or nephropathy in aged mice was also shown to be statistically significant (p. 0323). Some mice that demonstrated symptoms of arteritis or GN also developed anemia. Found were two Fus1^{+/-} and three Fus1^{-/-} mice that showed a considerably reduced red cell count (RBC), as opposed to only one such case in WT mice. Moreover, two Fus1-deficient mice but none of the WT cohort showed a dramatically increased white blood cell count (WBC). All these mice had arteritis and/or GN. An association between anemia or high WBC with arteritis and/or severe GN with tubular casts was noticed in the Fus1^{+/-} and Fus1^{-/-} mice that died prematurely. Notably, one Fus1^{+/-} and two Fus1^{-/-} mice presenting with a vasculitis syndrome also developed fat necrosis, a condition often associated with certain types of vasculitis (14) or systemic lupus erythomatosis (SLE) (15). One aged Fus1^{+/-} mouse showed signs of atherosclerosis of the lung artery, a very rare event for WT mice.

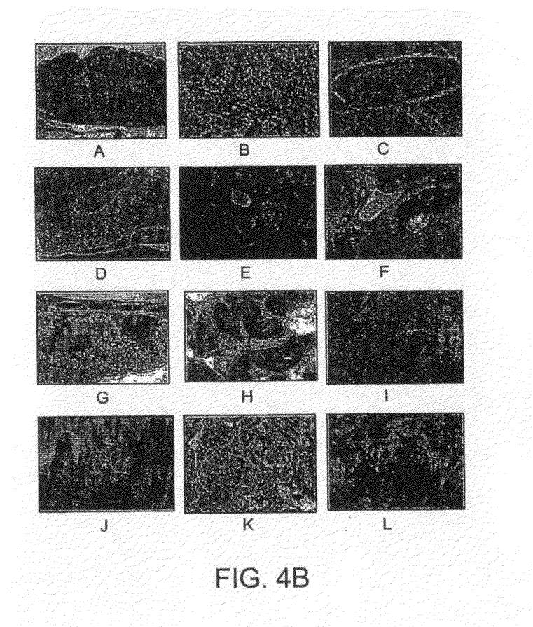

Because Fus1^{-/-} mice exhibited histopathological signs and phenotypic characteristics of an autoimmune disease, these animals were tested for circulating autoimmune Abs specifically found in autoimmune disorders, such as SLE (16) or certain types of vasculitis (17). Indeed, circulating autoreactive nuclear Abs were found in Fus1^{+/-} and Fus1^{-/-} mice (FIG. 4A). Taken together, these results indicate that Fus1 gene deficiency results in the development of primary systemic vasculitis with a rather median or late onset and many symptoms typical for SLE.

### Localization and Nature of Vasculitis Lesions and Glomerulonephritis

Arteritis was found in large vessels as well as in small arteries, veins and capillaries. Multiple organs, such as heart (the aorta and its primary and secondary branches), kidney, lung, liver, pancreas, spleen, lymph nodes, thyroid, gall bladder, tongue, spinal cord muscles and omentum were affected. Some mice developed fibrinoid necrotizing arteritis in the following organs: pancreas, liver, spleen, small intestine, ovary, uterus, urethra and spinal cord. Small and medium-size arteritis affecting multiple organs, especially the skin, peripheral nerves, gut, kidney and heart, is a feature of polyarteritis nodosa. Fibrinoid necrosis and destruction of the arterial wall are also hallmarks of non-granulomatous vasculitides, such as polyarteritis nodosa and microscopic polyangiitis as opposed to giant-cell arteritis that lacks necrosis (18). FIG. 4B illustrates some typical arteritic lesions that were found in Fus1^{+/-} and Fus1^{-/-} mice. An inflamed coronary artery (Panel A) was found surrounded by a massive cellular infiltrate. A higher magnification (Panel B) revealed the characteristic whorled structure of chronic inflammation, the arrow indicating the leukocytes (L) adhering to the endothelium (E). The light microscopic appearance of fibrinoid necrotizing arteritis is similar to that of microscopic polyangiitis and Wegener granulomatosis. Panels D and E show typical necrotizing arteritis lesions observed in the pancreas that display massive infiltration of the entire vessel and sub-endothelial fibrinoid necrosis. For comparison, control pancreatic vessels are depicted in panel H. The collapse and stenosis or ballooning and rupture of affected vessels probably results from the destruction of the elastic laminae. Panels J and K represent omentum arteries stained for elastin and connective tissue. The rightmost portion of the vessel was unaffected, as the elastic layers remain intact (Panel J). The left portion of the vessel was severely inflamed, with a massive trans-mural infiltrate of leukocytes and destruction of elastin layers. In panel K, both elastin layers are affected. Moreover, the loose connective tissue of the tunica adventia (in red), that should closely surround the normal vessel, is dispersed by the infiltration around the inflamed vessel. Glomerular lesions, most often very heterogeneous, included membranoproliferative changes, wire-loop-like subendothelial deposits and voluminous intracapillari trombi of PAS-positive material, often obstructing the glomerular capillary lumen and sometimes, glomerular sclerosis associated with extensive tubular cast formation (Panel K and L).

Fus1-Deficient Mice have Increased Susceptibility to a Certain Range of Tumors

Fus1^{+/-} and Fus1^{-/-} mice revealed a tendency to develop malignancies and die earlier than the age-matched cohort of WT mice. Thus, at the age of 6-13 months there was no incidence of lymphoma recorded in WT mice, while two Fus1^{+/-} mice and one Fus1^{-/-} mouse developed various lymphoma types at the respective ages of 8, 12 and 7 months. One Fus1^{-/-} mouse developed an invasive squamus cell skin carcinoma at the age of 13 months (Table I). To compare the frequency of tumor incidence in old mice, 27 WT, 39 Fus1^{+/-} and 25 Fus1^{-/-} mice were monitored over a 2-year period, at which time all mice were sacrificed. The most prevalent malignancy as compare to the WT mice was hemangioma/hemangiosarcoma (Table II): 8/25 (32%) Fus1^{-/-} mice and 9/39 (˜23%) Fus1^{+/-} mice developed a mixed hemangioma/hemangiosarcoma or either of them independently, as compared to 1/27 (4%) WT mice. Fus1^{-/-} females were most prone to the development of these vascular tumors. A Fisher's exact test showed a statistically significant relation between the Fus1 disruption and the development of vascular tumors (p=0.0178). Although proliferative vascular lesions in the Fus1^{+/-} and Fus1^{-/-} mice were of highest incidence in the spleen and the liver, they were also found in other organs (Table III). Fus1-deficient mice also demonstrated an increase in lymphoma frequency from 22% in WT to 33% in knockout mice (Table III), though the relation between the disruption of Fus1 and the development of lymphoma was not statistically significant (p=0.6084). Follicular center cell (FCC) lymphoma represents the predominant type found in WT and Fus1-deficient mice. However, other types of lymphoma were found in the Fus1-deficient mice: one splenic marginal zone, one lymphoblastic, and two mixed FCC lymphomas. In addition, one Fus1^{+/-} female presented an erythroleukemia. No distinct tumor pattern was observed in the organs outside the hematopoietic system (Table III). The fact that Fus1^{+/-} and Fus1^{-/-} mice possess a similar frequency of tumor incidence suggests that Fus1 acts as a recessive tumor suppressor.

### Exploration of the T and B Cell Compartments of the Fus1-Deficient Mice

T and B cell populations have been investigated by flow cytometry in 6-8 month old Fus1^{-/-}, Fus1^{+/-} and WT mice. The percentage of lymphocytes expressing lineage-specific cell surface markers such as CD3, CD4, CD5, CD8, CD25, and CD44 were evaluated for the T cell compartment, and CD19, B220 and IgM for the B cell population. No major differences were found in the thymus for the T cell compartment, considering either the mature (CD4/CD8) or the immature (CD3⁻CD25/CD44) T cell subpopulations. Flow cytometry analysis of T and B lymphocytes isolated from lymph nodes and spleen did not reveal any significant differences in their activation status i.e. the expression of CD25, CD44 (T cell), and CD69 (B cells) (data not shown). These data suggest that Fus1 does not play an essential role in T or B cell development or in the homeostasis of these peripheral cell populations.

### The Humoral Response in Fus1-Deficient Mice

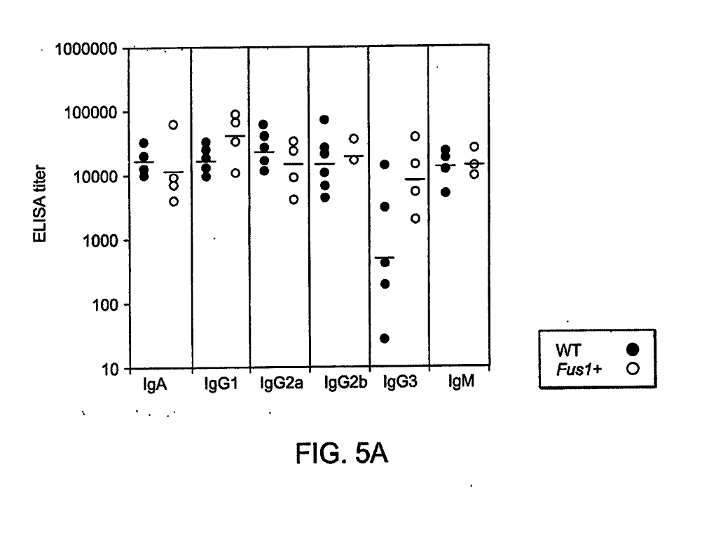

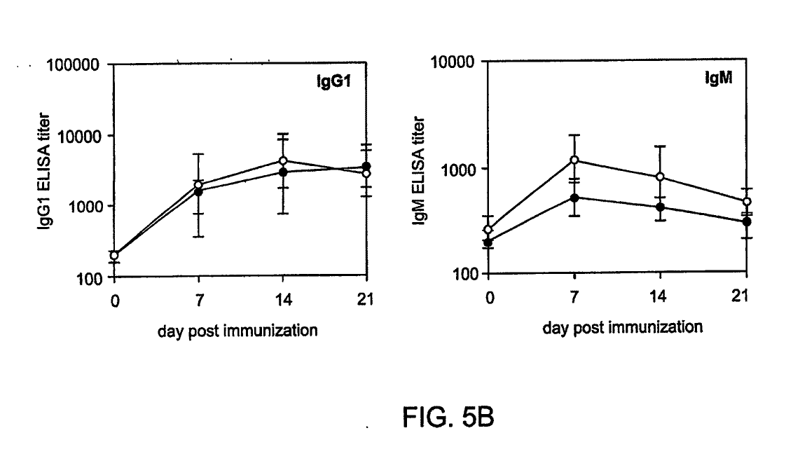

To assess the role of Fus1 in Immunoglobulin (Ig) class switching, the basal serum levels of different isotypes observed in non-immunized mice were compared. Levels of IgA, IgG1, IgG2a, IgG2b, IgG3 and IgM were not statistically significantly different in Fus1-deficient mice at 8 months of age (data not shown). Similarly, no differences were observed for the basal levels of IgG2a, IgG2b and IgM at 12 months of age (FIG. 5A). However, the majority of Fus1^{-/-} mice present a higher average level of IgG3. In addition, 4/5 Fus1^{-/-} mice tested possess a lower level of IgA and an increased level of IgG1 compared to WT mice (FIG. 5A). The ability to generate a T cell-dependent antibody response in Fus1^{-/-} mice was assessed by measuring the in vivo response after a challenge with trinitrophenyl-keyhole limpet hemocyanin (TNP-KLH) in alum. No significant difference in the IgM/IgG1 Ag-specific profile was observed between the Fus1^{-/-} and the control mice (FIG. 5B). This suggests that Fus1^{-/-} mice do not present any intrinsic defect in the Ab production. The observed increase in the total Ig level in the 12-month-old mice could be related to the development of autoimmunity.

### Fus1 Deficiency Affects the Maturation of NK Cells in Fus1^{-/-} Mice

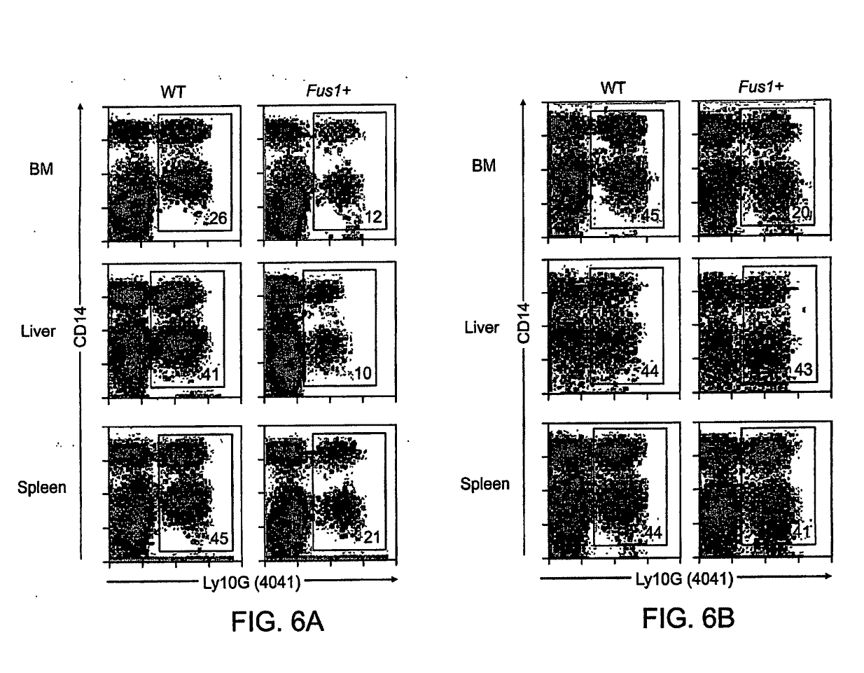

The NK cell compartment present in the bone marrow, liver, and spleen of 4 week old Fus1^{-/-} and WT mice were explored. Freshly isolated lymphocytes were analyzed by four-color flow cytometry (Table IV). Observed were at least a 45% decrease of the total number of NK cells as compared to the control, that is statistically different in the bone marrow (˜45%, p=0.0317) and the spleen (˜56% p=0.0079) but not in the liver (˜46%, p=0.9520). However, the evaluation of the percentage of CD3⁻DX5⁺ NK cells in Fus1^{-/-} mice did not reveal a statistically significant difference (respectively: p=0.3095, p=0.3095, p=0.8413). As the same observation was made for the T cell compartment (data not shown), it seems that it is the size of the whole lymphocyte compartment that is affected and not an intrinsic defect of NK cell generation. The expression of two receptors for MHC class I were than investigated that are expressed different stages of NK cell maturation (19-21). Ly49G belongs to the multigenic and polymorphic family of Ly49 receptors and represents an evolutionary conserved allele shared between the 129/J and B6 strains (22). A statistically significant decrease of the percentage of NK cells expressing Ly49G in the liver (˜57%, p=0.0079) and the spleen (˜35%, p10159) of Fus1^{-/-} mice as compared to WT mice (Table N, FIG. 6A) were observed, suggesting a decrease in the size of the mature CD94⁺Ly49G⁺ NK cell compartment. There was a ˜32% decrease of this subpopulation in bone marrow though the difference was not statistically significant (p=0.5714). In contrast, there was no statistically significant alteration in the expression of CD94 on NK cells in any tissue, i.e. bone marrow (p=0.1508), liver (p=0.3095) or spleen (p=0.1508). Taken together, these data suggest a significant delay in NK cell maturation characterized by a blockade at the immature CD94^{bright}Ly49⁻ developmental stage in Fus1^{-/-} mice.

### Low Levels of IL-15 in Fus1-Deficient Mice

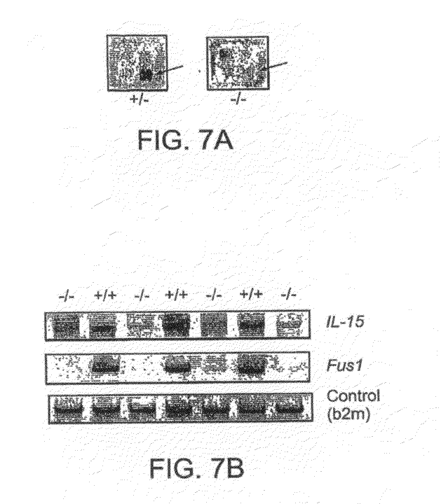

A number of cytokines have been implicated in the development and function of NK cells (23). These include IL-2, IL-7, IL-12, IL-15, and IL-18. Accordingly, the expression of these and other cytokines in the Fus1^{-/-} mice was studied. The GEArray® Q Series Mouse Common Cytokines Gene Array (SuperArray, Frederick, Md.) that contains 96 common cytokine genes was used. Side-by-side hybridization of SuperArray membranes with cDNA probe generated on mRNAs isolated from the bone marrow or spleen of Fus1^{-/-} and WT mice demonstrated that the only cytokine consistently down-regulated in Fus1^{-/-} was IL-15 (FIG. 7A). Quantitative RT-PCR on the bone marrow mRNA from three sets of Fus1^{-/-} and WT mice confirmed the IL-15 down-regulation in the Fus1-deficient mice (FIG. 7B). IL-15 is thought to be critical for early NK cell differentiation by maintaining normal numbers of immature and mature NK cells (9, 23) and it is involved in the expression of Ly49 receptors on NK cells (24). Thus, the defect in IL-15 production could be responsible for the delay in NK cell maturation. In addition, as acquisition of CD94 and Ly49G occurs before the expansion stage, this data suggest that the decrease in the total number of NK cells per lymphoid tissue in Fus1^{-/-} mice is related to the lower level of IL-15. The same observation can apply to the T cell compartment as IL-15 is also involved in T cell development (25, 26).

### IL-15 Restores the Mature Phenotype of NK Cells in Fus1-Deficient Mice

To investigate if the delay in NK cell maturation was the consequence of an intrinsic NK cell defect or related to the lack of IL-15 production, 5 μg of pCMV-SPORT6 IL-15 expression vector was introduced by injection into tail vein of Fus1^{-/-} mice and the effect on the NK cell compartment was analyzed four days post-injection. Observed was a complete restoration of the mature NK phenotype in Fus1^{-/-} liver and spleen (Table IV, FIG. 6B). No statistically significant difference was detected between the percentages of Ly49 expressing NK cells in Fus1^{-/-} mice as compared to WT mice (respectively p=0.9372 p=0.8182). Only a partial recovery was noticed for the NK cell compartment in the bone marrow (p=0.0022). These results suggest that Fus1 deficiency affects NK cell maturation through the reduction of IL-15 production but does not directly alter their developmental process.

The inactivation of Fus1 in mouse generates the development of characteristic signs of autoimmune disease, such as circulating autoimmune Abs, arteritis, and GN. Using expression array analysis, an insufficient production of IL-15 in Fus1-deficient bone marrow was identified. Finally, this links an IL-15 deficit with the observed defect in NK cell maturation.

Essential mediators of innate defense, NK cells can rapidly recognize and destroy infected or malignant cells, as well as modulate the activity of other immune cells via cytokine and chemokine production. NK cells also play a decisive role in the regulation of adaptive immunity by stimulating other components of anti-tumor immune response (27). Numerous studies have emphasized the major role of IL-15 in NK cell development (9, 28, 29). IL-15 is produced by the bone marrow stroma cells and induces NK cell differentiation from mouse haematopoietic pluripotent cells (HPC) even in the absence of other cytokines (30). Studies on IL-15 (11), IL-15 receptor (10, 31) or IRF-1-deficient mice (32, 33) also proved the direct involvement of IL-15 in NK cell differentiation and proliferation. In the study, young (4-6 week old) Fus1^{-/-} mice showed significant reduction in mature NK cells as compared with WT littermates. Injection of an IL-15 expression construct in null mutants rescued this deficiency, strongly supporting the proposed link between Fus1, IL-15 expression regulation and NK cell maturation.

The analysis of TNP-specific humoral immune response in Fus1-deficient mice showed a normal T-dependent response including Ig class-switching.

Autoimmune disease is one of the major features distinguishing Fus1^{-/-} and IL-15-deficient mice. Indeed, while both models display marked reduction in peripheral NK cells combined with normal development of lymphoid organs and unchanged Ig levels, no signs of immunodeficiency have been reported in IL-15^{-/-} animals (11). The association between NK cell deficiency and autoimmune disease, however, was previously documented for multiple sclerosis (MS), type I diabetes, and SLE. Patients with these diseases commonly show low levels of peripheral blood NK cells (36, 37). Defects in NK cell activity have been also reported in several animal models of autoimmunity, such as lpr mice, a model of SLE; EAE (experimental allergic encephalitis) mice, a model of MS; and NOD mice, a model of type I diabetes and SLE. In each of these models, strong evidence of an NK cell immunoregulatory role has been obtained (37). Thus, the observed hallmarks of autoimmunity in the Fus1 model may be directly related to the NK maturation defect, and scrutinizing this model may provide valuable data on cellular and molecular mechanisms of this association.

Incomplete penetrance of autoimmunity in the Fus1 model appears to be related to the background genes. Whereas the NK cell maturation defect was observed in 100% of Fus1-deficient animals analyzed, three major groups of Fus1-deficient phenotypes were defined. The first group was distinguished with vasculitis, glomerulonephritis, and blood cell abnormalities; the second group had either GN or vasculitis; the third group, representing the majority of mice, displayed no visible signs of autoimmune disease. About 70% of Fus1^{-/-} animals produced autoantibodies. The observed heterogeneity of symptoms is characteristic for a multifactorial disease and thus argues for involvement of yet undefined genes, which may determine the ultimate manifestation of the disease.

A significant increase in spontaneous hemangioma/hemangiosarcoma tumors in aging Fus1^{+/-} and Fus1^{-/-} mice is one of the most remarkable features supporting the role of Fus1 as a tumor suppressor. Susceptibility of Fus1-deficient mice to vascular lesions and neoplasms may support the hypothesis that malignancies may arise from the areas of inflammation as a pathological consequence of the host response (reviewed in 38). Indeed, experimental evidence obtained in mice link microbe-associated or autoimmune chronic inflammation with cancer (39, 40). However, it is currently unknown if vascular tumors generated in the Fus1 model have an inflammatory origin. Spontaneous tumorigenesis in Fus1-deficient mice is different in tumor spectrum from WT mice of the same genetic background and may serve as an efficient tool for further elucidation of the role of NK cells in tumor rejection.

Additional evidence of Fus1 tumor suppressor activity comes from the increased incidence of hematopoietic malignancies in aging Fus1^{+/-} and Fus1^{-/-} mice (32% versus 22% in the WT cohort). Also observed were three cases of lymphomas in the Fus1-deficient mice of 7-12 months of age, while none was found in WT littermates of the same age. Even though FCC lymphoma was predominant in both WT and Fus1-deficient mice, the latter group was distinguished with somewhat rare types of hematopoietic malignancies: one splenic marginal zone, two lymphoblastic, two mixed FCC, and one erythroleukemia that were not observed in the WT cohort. Epidemiological data support the association between autoimmunity and malignant lymphomas (41). Transfer of the Fus1 model to a more favorable genetic background may provide a better tool for studying the association between NK cells and lymphomas.

Additional study is also warranted to elucidate the possible involvement of Fus1 in susceptibility to virus-associated malignancies. This category of tumors occurs at an unusually high rate in immunocompromised mice or in patients with immune system dysfunction. Since virus-associated tumors constitute about 15% of total tumor cases in humans (42), it would be of great interest to exploit the Fus1 model in this direction. Growth suppressor properties of FUS1 both in vitro (2) and in vivo (3) combined with the data on the role of Fus1 in innate immunity argue that this protein may possess, at least in humans, a pleiotropic tumor suppressor effect. Molecular mechanisms of its growth suppression activity remain to be elucidated.

The fact that mice heterozygous for the targeted Fus1 allele also showed increased susceptibility to malignant tumors suggests that complete loss of the normal Fus1 allele, according to the two-hit scenario (43, 44), is not necessarily required in this case. It would be of great interest to see if loss of one Fus1 normal allele has similar consequences in humans, since this haploinsufficiency effect may explain the low frequency of mutations (3 out of 79) observed in the remaining Fus1 allele in lung tumors (1). It would be also important to assess a possible link between Fus1 and hematopoietic malignancies in humans.

Pursuing the biological function of FUS1, the intracellular localization of the protein was analyzed. A preferential mitochondrial localization of FUS1 with a lesser amount of the protein localized in the ER was demonstrated. Confinement of the FUS1 protein to cytoplasmic organelles was also verified with the Abs against the endogenous protein (12). In silico studies presented in this manuscript are also in favor of the mitochondrial localization of the FUS1 protein. However, the possibility that under certain stimuli, Fus1 may be transported to nucleus in association with other proteins could not be excluded. PHI- and PSI-BLAST analysis (http://www.ncbi.nlm.nih.gov/BLAST/) of Fus1 homology showed that the C-terminal 60 amino acids of Fus1 are 40% homologous to the transcription factor IRF-7 (interferon regulatory factor 7) (45). The unpublished observations regarding Fus1 protein-partners indicates that protein-chaperons may facilitate Fus1 travel between different cellular compartments. These data imply that Fus1 has the potential to directly regulate IL-15 transcription.

The observed Fus1 effect on IL-15 expression also could be mediated through a yet unknown signal transduction pathway. Noteworthy, in silico analysis indicates that although the signal peptide was not determined, FUS1 could be secreted out of the cell by an unconventional mechanism. The potential ability of the FUS1 protein to be secreted is in line with the novel signal transduction pathway's hypothesis. The predominant mitochondrial localization of FUS1 is intriguing and warrants further investigation into the molecular mechanisms of FUS1 action.

Bioinformatics analysis of Fus1.

Bioinformatics analysis was compiled using web-based bioinformatics servers:

http://us.expasy.org/;

http://www.ncbi.nlm.nih.gov/; http://scansite.mit.edu/;

http://www.genome.jp/SIT/ploc.html; http://www.rcsb.org/pdb/;

http://www.sanger.ac.uk/Software/Pfam/; http://smart.embl-heidelberg.de/;

http://www.cbs.dtu.dk/services/; http://www.ebi.ac.uk/interpro/).

### Generation and Characterization of Fus1^{-/-} Mice

A 16.5 kb genomic DNA fragment containing the entire mouse Fus1 gene and adjacent sequences was isolated from 129/SvJ lambda FIXII library (Stratagene, La Jolla, Calif.). The neo-gene with the phosphor-glycerol kinase 1 promoter and the bovine growth hormone polyadenylation sequence (pGKneobpA) was employed as a positive selectable marker. The pGK-thymidine kinase cassette was used as a negative selectable marker (46). First, a 3.7 kb Nar/HindIII fragment containing a 5′ non-coding sequence of the first exon was inserted into the pJMM4-neo vector at the HindIII site. Next, a 2.1 kb EcoRI/HindIII fragment containing a 3′ non-coding region of the third Fus1 exon and a part of the adjacent Luca2 gene was ligated into the NotI site of the pJMM4-neo/3.7 kb Nar/HindIII containing plasmid. The resulting plasmid was linearized with SalI. Electroporation and selection were performed using CJ7 ES cell line, as described in Tessarolo et al. (47). Chromosomal DNA samples extracted from G418/FIAU-resistant ES clones were screened using diagnostic EcoRI restriction sites and a 2.1 kb probe 5′-external to the targeting sequence indicated in FIG. 3A. This probe detected a ˜8.5 kb EcoRI fragment in WT DNA and a 7.4 kb EcoRI fragment in the mutant allele. The Fus1-targeted cell line was injected into C57BL/6J blastocysts and chimeric mice were generated and selected as previously described (48). Selected chimeras that show germline transmission of the Fus1 knockout allele were then mated to C57BL/6J females to establish mouse lines.\

### Northern Blot Analysis

For Fus1 expression analysis, we used poly-A RNA samples isolated from mouse tissues using FastTrack® kit (Invitrogen, Carlsbad, Calif.). RNA was then fractionated in a formaldehyde agarose gel and the level of Fus1 transcription in mouse tissues was assessed by radioactive hybridization with a Fus1 cDNA probe. For analysis of Fus1 expression in mouse tissues we used MTN™ membrane, “Clontech”

### Comparative PCR and RT-PCR

Primers design and optimization of RT-PCR conditions were performed using GeneFisher Primer Calculator (http://www.genefisher.de). Number of PCR cycles was limited to 25-30 for a more accurate comparison. For PCR analysis of Fus1 expression in lymphoid and other mice tissues we used MTCII cDNA panel (Clontech, Palo Alto, Calif.), for analysis of FUS1, IL-15 and IL-2 expression in human blood fraction we used Human blood fractions cDNA MTC panel (Clontech, Palo Alto, Calif.).

### Western Blot

Nitrocellulose strips containing nuclear extract from mouse spleen were blocked for 1 hour in blocking buffer followed by incubation with serum (dilution 1:125) overnight at 4° C. The strips were washed and incubated with an HRP-conjugated anti-mouse IgG Ab (Santa Cruz Biotechnology, Inc, dilution 1:20 000) for 1 hr at RT. The strips were then washed and exposed to film using a chemiluminescent substrate (SuperSignal® West Pico Chemiluminescent substrate, Pierce).

### Cell Fractionation

293T cells transfected with the FUS1/FLAG construct were washed in ice cold PBS, scraped from the plates and collected by centrifugation at 400 g. Cells were then resuspended in 1 ml of hypotonic buffer (0.25M sucrose, 10 mM Tris-HCl, pH7.5) with protease inhibitors added and homogenized with Dounce Tissue Grinder. Disrupted cells were centrifuged at 600 g for 10 min. The nuclear fraction was obtained from the pellet by washing it with a hypotonic buffer with subsequent lysis in 1 ml of 1×SDS-sample buffer. The supernatant i.e. the post nuclear fraction was fractionated by centrifugation at 8000 g for 10 min. The mitochondrial fraction was obtained by adding 0.2 ml of 1×SDS-sample buffer to the pellet. The supernatant i.e. the post mitochondria fraction was removed and used as a cytoplasmic fraction. Western Blot analysis has been performed on 20 μl of each fraction.

### Immunostaining

293T cells were transiently transfected with the FUS1/FLAG construct. Twenty-four hours post-transfection cells were plated on coverslips and cultured overnight. Cultured cells were washed in PBS, fixed in 4-7% paraformaldehyde for 30 min and permeabilized with 0.1% Triton X-100 (Sigma) for 5 min. After a pre-incubation with 1% fetal bovine serum in DMEM (Gibco BRL), cells were stained with mouse anti-FLAG M2 Abs (1:500) (Sigma) followed by rabbit anti-cytochrome c Abs (1:250) (Santa Cruz Biotechnology, Inc.) or rabbit anti-PDI antibodies (1:500) (Calbiochem). The visualization of red and/or green signal was performed by using respectively Alexa Fluor™ 594-labeled goat anti-mouse (red) and Alexa Fluor 488-labeled anti-rabbit secondary (green) Abs (1:500) (Molecular Probes).

### In Vivo Immunization

Mice were immunized with a single intraperitoneal injection of 100 μg of TNP-KLH in Imject Alum (Pierce Chemicals Co). The serum samples were collected on day 0 (before immunization), days 7, 14 and 21 (after immunization) and Ag-specific serum levels of 1 g were assayed by ELISA.

### ELISA

Total IgA, IgG1, IgG2a, IgG2b, IgG3, or IgM were captured with purified goat anti-mouse IgA, IgG1, IgG2b, IgG3 or IgM and detected with HRP-conjugated goat antimouse α, α1, α2a, α2b, α3, or α(SouthernBiotech). To measure Ag-specific Ig, plates were coated with 2.5 μg/well TNP-OVA, and Ig was detected with HRP-conjugated anti-α or α1. The reaction was developed by using ABTS® Microwell Peroxidase Substrate System (KPL Inc.) with optic density measured at 405 nm. Titers were determined by the interpolation of the dilution of serum that gave a 50% OD of the maximum absorbance achieved.

### Antibodies

The following fluorochrome-conjugated monoclonal Abs (mAb) used in this study were purchased from Pharmingen: FITC-anti-Ly49G2 (rIgG2a, 4D11), FITC- or PE-anti-CD4 (rIgG2b, L3T4), FITC- or PE-anti-CD8a (rIgG2a, 53-6.7), FITC-anti-CD19 (rIgG2a, 1D3), PE-anti-CD69 (hIgG1, H1.2F3), APC- or PerCP-anti-CD3α(hIGg1, 145-2C11), APC-anti-C62L (rIgG2a, MEL-14), PE-Cy5-anti-CD44 (rIgG2b, IM7) and APC-Cy7-anti-CD25 (rIgG1, PC61). PE-anti-CD94 (rIgG2a,18d3) and APC-anti-CD49b (rIgM, DX5) had been purchased at eBioscience.

### Cell Preparation and Flow Cytometry

Single-cell suspensions were prepared from various tissues such as thymus, spleen, liver, bone marrow and lymph nodes, and depleted of red blood cells. Absolute numbers of cells were derived from total lymphocyte count obtained from an automatic hemocytometer Sysmex KX-21 (Roche Diagnostics). For flow cytometry, the cells were stained with combinations up to 5 of the indicated fluorochrome-conjugated mAbs. Stained cells were analyzed with a FACScalibur or a FACSort™ (Becton Dickinson). B cells are represented by the CD19 expressing lymphocytes. Resting mature and immature T cells were distinguished by expression of CD3, CD4, CD8, CD44 and CD25. Thus, the most immature T cells are represented by the CD3⁻CD4⁻CD8⁻CD25⁺CD44⁺ lymphocytes (Double negative 1 or DN1). Differentiation to a more mature stage proceeds via the following steps: CD3⁻CD4⁻CD8⁻CD25⁺CD44⁺ (DN2), CD3⁻CD4⁻CD8⁻CD25⁺CD44⁻ (DN3), CD3⁻CD4⁻CD8⁻CD25⁻CD44⁻ (DN4), CD3⁺CD25⁻CD44⁻CD4⁺CD8⁺ (Double Positive or DP), and finally CD3⁺CD25⁻CD44⁻CD4⁺CD8⁻ (Simple Positive or SP CD4⁺ T cells) or CD3⁺CD25⁻CD44⁻CD4⁻CD8⁺ (SP CD8⁺ T cells). CD25, CD62L and CD69 expression on T and B cells was explored to detect a potent activation status of these lymphocyte subpopulations in Fus1-deficient mice. NK cells were defined as CD3⁻DX5⁺ lymphocytes. WinMDI 2.8 (TRSI) and CellQuest (Becton Dickinson) softwares were respectively used for acquisition and analysis of data.

### Analysis of Cytokine Expression Using SuperArray Membranes

Probes for expression analysis were generated on total RNAs isolated from BM of WT and Fus1^{-/-} mice with RNeasy Kit (Quagen, Valencia, Calif.). GEArray® Q Series Mouse Common Cytokines Gene Arrays representing 96 common cytokine genes and AmpoLabeling-LPR kit were purchased from SuperArray (Frederick, Md.). Radiolabeling and hybridization procedures were done according to the manufacturer's protocol.

### Induction of IL-15 Production

5 μg of a pCMV6-SPORT expression vector containing the entire mouse IL-15 cDNA in 100 μl of distilled water were injected into the tail vein. Four days post-injection mice were sacrificed and NK cells from bone marrow, liver and spleen were analyzed as described above.

### Statistical Analysis

Fisher exact test was performed using GraphPad Quickcalcs Server at http://www.graphpad.com/quickcalcs/index.cfm. Statistical analysis of WT and Fus1^{-/-} NK cell compartment distributions were obtained using GraphPad Prism software (San Diego, Calif.). Comparison of distributions was performed using a Mann-Whitney U-test. A p value <0.05 was considered as significant.

### FUS1 Genome Sequence

A 1864 bp NarI/HindIII mouse DNA fragment containing the FUS1 1 gene (pos. 175010 to 173147 on GenBank AC025353, reverse complement) was substituted with the neo gene using a 5′-(HindIII/NarI, 3501 bp, pos. 178510-175010, reverse complement) and a 3′-flanking (HindIII/EcoRI, 2098 bp, pos. 173147-171049, reverse complement) mouse DNA sequences. The neo gene with the phosphor-glycerol kinase 1 promoter and the bovine growth hormonepolyadenylation sequence (pGKneobpA) was employed as a positive selectable marker; the pGK-thymidine kinase cassette was used as a negative selectable marker (Bonin et al: Isolation, microinjection, and transfer of mouse blastocysts. Methods Mol Biol 2001, 158:121-134).

### FUS1 1 Orthologs

FUS1_Human
(SEQ. ID. NO.: 1)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEV
Fus1_Mouse
(SEQ. ID. NO.: 2)
MGASGSKARG LWPFASTPGG GGPEAAGSEQ SLVRSRARAV
PPFVFTRRGSMFYDEDGDLAHEFYEETIVT KNGQKRAKLR
RVHKNLIPQG IVKLDPPRIH VDFPVILYEV
Fus1_Rat
(SEQ. ID. NO.: 3)
MGSSISKARGLCPFVSTTGVGSPVAEVAKQSLVRSRARAVPPFVFTRRG
SMFYDEDGDLAHEFYEETIITKNGQKRSNLRRVRNNLIPQGIVKLERPQ
IHVDFPLILCEV
Fus1_Zebra
(SEQ. ID. NO.: 4)
MGGSGSKGKGYWPFSGSGGGDEPAKEGQEQSLSRVRSIRNATPFVFTRR
SSLYFDEDGDLAHEFYEETVVTKNGRKKAKLKRIHKNLIPQGIIKLDHP
CIHVDFPVVICEA
Fus1_Xenopus laevis
(SEQ. ID. NO.: 5)
MGGSASKARG LWPFSSTTSE AQPGNDDQSV TRMRKATPFI
FTRRGSMYFD EDGDLAHEFY EETVVMKNGR KRAKLKRIQK
NLRPQGIIRL DHPCLHVDFP VVICEV
Fus1_C. Elegans
(SEQ. ID. NO.: 6)
MGLGSSKRKE EPPHKSEPKT VGRVKRAGAR PDEMIAKYAE
VLKTRGILPE YFLVHEAKSSQYIDEDGDVA NEFYQETMSD
GEKRRLCRLM KNLRPKGKER YAIPRLKHDI PVVIWEVQQPQET
FUS1_Human [Homo sapiens]
(SEQ. ID. NO.: 7)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEV

### Peptide Fus1 Variants—FUS1 Splice Variants:

```
Peptide Sequence FUS1.aDec03 110 AA
(SEQ. ID. NO.: 8)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEV
Peptide Sequence FUS1.dDec03 96 AA
(SEQ. ID. NO.: 9)
LRCEQSGHGARHAPAALTWAPAGPKLGACGPSPRRPEAAAQRQQELSKL
WCGLGAELCPPSYSRAAALCSMMRMGIWLTSSMRRQSSPRTGRSGPS
Peptide Sequence FUS1.eDec03 81 AA
(SEQ. ID. NO.: 10)
MAALAEQIKDENWPWWLPGCSMFYDEDGDLAHEFYEETIVTKNGQKRAK
LRRVHKNLIPQGIVKLDHPRIHVDFPVILYEV
Peptide Sequence FUS1.FDec03 80 AA
(SEQ. ID. NO.: 11)
LNKSRMRTGLGGCLDGVLCSMFYDEDGDLAHEFYEETIVTKNGQKRAKL
RRVBKNLIPQGIVKLDHPRIHVDFPVILYEV
Accession No.: AF055479.
Homo sapiens lung FUS1 (FUS1)
(SEQ. ID. NO.: 12)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEV
Accession No.: AF055479
(SEQ. ID. NO.: 14)
      1 tgcggccgcg tttccgtgga gacagccgag cctgcggaag
          gcggcggcgg cggcacctgc
    61 gatcagcggc tggggcaggt tatggtagtg cggactgcgg
          tgtgagcaga gcggccacgg
  121 ggcccgccat gcgccggcgg ccctgacatg ggcgccagcg
          ggtccaaagc tcggggcctg
  181 tggcccttcg cctcggcggc cggaggcggc ggctcagagg
          cagcaggagc tgagcaagct
  241 ttggtgcggc ctcggggccg agctgtgccc cccttcgtat
          tcacgcgccg cggctctatg
  301 ttctatgatg aggatgggga tctggctcac gagttctatg
          aggagacaat cgtcaccaag
  361 aacgggcaga agcgggccaa gctgaggcga gtgcataaga
          atctgattcc tcagggcatc
  421 gtgaagctgg atcacccccg catccacgtg gatttccctg
          tgatcctcta tgaggtgtga
  481 ccctgggagg tggcagacag aagcaccccc tgccccggca
          agaaactccc aggctcaatc
  541 aaggtgtggc ttccattgag gagcccaggc tggggccaca
          accctgaata aactctgttg
  601 gcccataacc ttcagctgtg agcgggtcgg tcccacagta
          ttggttgggt gttggtttgt
  661 gtgtggacaa gaggtggttg gtgggtggtg aaggctaatg
          gcagagttag caccccactc
  721 tcccaagcca cccctgcaag cagcatagca gggcatatac
          cagtcaggaa tgcccgttac
  781 ctggttcctt gcctggtctg ctttcttcca agtttgcctg
          gggcctagcc ctgctagagg
  841 ctacagcact ttacaagcaa ggtatgcttt cttccagccc
          ctaggctgtg ggcactgtat
  901 acaagtagga acttcctttc cttcacttcc cttttaaccc
          ctagtcagag catttcagcc
  961 gtttgctacc tcgattcctc ctgtgttgga cagaggctgg
          gggcagtgcc agcctgattc
1021 ttccgaccta cctgccattt gttcccgcct tcagatggat
          ggacagtttg ctggctattg
1081 ataggagtgg ggactgggtg ggggcttctc cctctaccca
          gggctgggct gatcccccta
1141 ctgcaactaa ctgttgcccc ccaaccccga acccccagtt
          gaggagttga gagagtgcag
1201 gctggggtca ggacaggctg cggatgcttg tgcctatggg
          gagttactcc aacccaccta
1261 ttctgtctaa tctccatggc tttgcaccaa atcctccacc
          cctccaattg ggaggggact
1321 gttcaccacc ttgtggtaag ggacaacacc ctaaggctgg
          tgccagtagt tatgagtagc
1381 ctaccacccc ctcccttaca gtaaccccca ccccttcagg
          atcagtcaag ggaaagcact
1441 agaacccctg ggtagggaaa gaaaggaggg aaaaaccata
          aaaggaatac ttataatgtg
1501 aaggtttgta aatagtccat gatgatgtcg tggcagagtc
          tgatttctat atagaggtga
1561 cttttttttt aagtactgtg caagctctgt gcttctataa
          tgtgggaaat ggcttgggga
1621 ggatggcccc tagcttagga agactgttgt gttatttgtt
          caatttcaat aaaatgattt
1681 gtagatcctg c
FUS1: Accession No.: BC023976. Homo sapiens, lung
(SEQ. ID. NO.: 13)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEV′′
Fus1: Accession No.: BC023976. Homo sapiens, lung
(SEQ. ID. NO.: 15)
      1 ggcacgaggc tgcgatcagc ggctggggca ggttatggta
          gtgcggactg cggtgtgagc
    61 agagcggcca cggggcccgc catgcgccgg cggccctgac
          atgggcgcca gcgggtccaa
  121 agctcggggc ctgtggccct tcgcctcggc ggccggaggc
          ggcggctcag aggcagcagg
  181 agctgagcaa gctttggtgc ggcctcgggg ccgagctgtg
          ccccccttcg tattcacgcg
  241 ccgcggctct atgttctatg atgaggatgg ggatctggct
          cacgagttct atgaggagac
  301 aatcgtcacc aagaacgggc agaagcgggc caagctgagg
          cgagtgcata agaatctgat
  361 tcctcagggc atcgtgaagc tggatcaccc ccgcatccac
          gtggatttcc ctgtgatcct
  421 ctatgaggtg tgaccctggg aggtggcaga cagaagcacc
          ccctgccccg gcaagaaact
  481 cccaggctca atcaaggtgt ggcttccatt gaggagccca
          ggctggggcc acaaccctga
  541 ataaactctg ttggcccata accttcagct gtgagcgggt
          cggtcccaca gtattggttg
  601 ggtgttggtt tgtgtgtgga caagaggtgg ttggtgggtg
          gtgaaggcta atggcagagt
  661 tagcacccca ctctcccaag ccacccctgc aagcagcaca
          gcagggcata taccagtcag
  721 gaatgcccgt tacctggttc cttgcctggt ctgctttctt
          ccaagtttgc ctggggccta
  781 gccctgctag aggctacagc actttacaag caaggtatgc
          tttcttccag cccctaggct
  841 gtgggcactg tatacaagta ggaacttcct ttccttcact
          tcccttttaa cccctagtca
  901 gagcatttca gccgtttgct acctcgattc ctcctgtgtt
          ggacagaggc tgggggcagt
  961 gccagcctga ttcttccgac ctacctgcca tttgttcccg
          ccttcagatg gatggacagt
1021 ttgctggcta ttgataggag tggggactgg gtgggggctt
          ctccctctac ccagggctgg
1081 gctgatcccc ctactgcaac taactgttgc cccccaaccc
          cgaaccccca gttgaggagt
1141 tgagagagtg caggctgggg tcaggacagg ctgcggatgc
          ttgtgcctat ggggagttac
1201 tccaacccac ctattctgtc taatctccat ggctttgcac
          caaatcctcc acccctccaa
1261 ttgggagggg actgttcacc accttgtggt aagggacaac
          accctaaggc tggtgccagt
1321 agttatgagt agcctaccac cccctccctt acagtaaccc
          ccaccccttc aggatcagtc
1381 aagggaaagc actagaaccc ctgggtaggg aaagaaagga
          gggaaaaacc ataaaaggaa
1441 tacttataat gtgaaggttt gtaaatagtc catgatgatg
          tcgtggcaga gtctgatttc
1501 tatatagagg tgactttttt tttaagtact gtgcaagctc
          tgtgcttcta taatgtggga
1561 aatggcttgg ggaggatggc ccctagctta ggaagactgt
          tgtgttattt gttcaatttc
1621 aataaaatga tttgtagatc ctgcaaaaaa
          aaaaaaaaaa aaaaaa
```

### Example 2

It should be understood that sequenced listed or claimed herein are meant to include fragments or variants thereof. For example, where listed or claimed, it should be understood to include sequences that share e.g., 65%, 70%, 75%, 80%, 85%, 90%, or 95% identity over a specified region.

To produce Fus1 TAT PTD-fusion proteins on either end of the Fus1 protein two oligonucleotides were synthesized and annealed to generate a double-stranded oligonucleotide with restriction sites chosen for convenient restriction enzymes and encoding the 11 amino acids (YGRKKRRQRRR of TAT PTD) from the basic domain of HIV Tat. The sequences were:

(SEQ ID NO: 18)
5′-XXXXCCTACGGCCGCAAGAAACGCCGCCAGCGCCGCCGCA-3′
and
(SEQ ID NO: 19)
5′-YYYYGCGGCGGCGCTGGCGGCGTTTCTTGCGGCCGTAGG-3′.

The fusion proteins and the surface accessibility (showing the presence of the PTD on the surface using the Motif Scanner server: http://scansite.mit.edu/motifscan_seq.phtml were given below:

### FUS1 Proteins:

Human:
(SEQ ID NO: 20)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEV

PTD-TAT Derivatives:

### (a) At the N-end

(SEQ ID NO: 21)
YGRKKRRQRRRMGASGSKARGLWPFASAAGGGGSEAAGAEQALVRPRGR
AVPPFVFRRGSMFYDEDGDLAHEFYEETIVTKNGQKRAKLRRVHKNLIP
QGIVKLDHPRIHIVDFPVILYEV

### At the COOH end

(SEQ ID NO: 22)
MGASGSKARGLWPFASAAGGGGSEAAGAEQALVRPRGRAVPPFVFTRRG
SMFYDEDGDLAHEFYEETIVTKNGQKRAKLRRVHKNLIPQGIVKLDHPR
IHVDFPVILYEVRRRQRRKKRGY

The 11-residue (YGRKKRRQRRR) TAT PTD sequence (SEQ ID NO: 23).

```
IL-15 Accession No.: BC018149
(SEQ ID NO: 16)
MRISKPHLRSISIQCYLCLLLNSHFLTEAGIHVFILGCFSAGLPKTEANWVNVISDLKKIED
LIQSMHIDATLYTESDVHPSCKVTAMKCFLLELQVISLESGDASIHDTVENLIILANNSLSS
NGNVTESGCKECEELEEKNIKEFLQSFVHIVQMFINTS
IL-15 Accession No.: BC018149
(SEQ ID NO: 17)
      1 actccgggtg gcaggcgccc gggggaatcc cagctgactc gctcactgcc ttcgaagtcc
    61 ggcgcccccc gggagggaac tgggtggccg caccctcccg gctgcggtgg ctgtcgcccc
  121 ccaccctgca gccaggactc gatggaggta cagagctcgg cttctttgcc ttgggagggg
  181 agtggtggtg gttgaaaggg cgatggaatt ttccccgaaa gcctacgccc agggcccctc
  241 ccagctccag cgttaccctc cggtctatcc tactggccga gctgccccgc cttctcatgg
  301 ggaaaactta gccgcaactt caatttttgg tttttccttt aatgacactt ctgaggctct
  361 cctagccatc ctcccgcttc cggaggagcg cagatcgcag gtccctttgc ccctggcgtg
  421 cgactcccta ctgcgctgcg ctcttacggc gttccaggct gctggctagc gcaaggcggg
  481 ccgggcaccc cgcgctccgc tgggagggtg agggacgcgc gtctggcggc cccagccaag
  541 ctgcgggttt ctgagaagac gctgtcccgc agccctgagg gctgagttct gcacccagtc
  601 aagctcagga aggccaagaa aagaatccat tccaatatat ggccatgtgg ctctttggag
  661 caatgttcca tcatgttcca tgctgctgac gtcacatgga gcacagaaat caatgttagc
  721 agatagccag cccatacaag atcgtattgt attgtaggag gcatcgtgga tggatggctg
  781 ctggaaaccc cttgccatag ccagctcttc ttcaatactt aaggatttac cgtggctttg
  841 agtaatgaga atttcgaaac cacatttgag aagtatttcc atccagtgct acttgtgttt
  901 acttctaaac agtcattttc taactgaagc tggcattcat gtcttcattt tgggctgttt
  961 cagtgcaggg cttcctaaaa cagaagccaa ctgggtgaat gtaataagtg atttgaaaaa
1021 aattgaagat cttattcaat ctatgcatat tgatgctact ttatatacgg aaagtgatgt
1081 tcaccccagt tgcaaagtaa cagcaatgaa gtgctttctc ttggagttac aagttatttc
1141 acttgagtcc ggagatgcaa gtattcatga tacagtagaa aatctgatca tcctagcaaa
1201 caacagtttg tcttctaatg ggaatgtaac agaatctgga tgcaaagaat gtgaggaact
1261 ggaggaaaaa aatattaaag aatttttgca gagttttgta catattgtcc aaatgttcat
1321 caacacttct tgattgcaat tgattctttt taaagtgttt ctgttattaa caaacatcac
1381 tctgctgctt agacataaca aaacactcgg catttcaaat gtgctgtcaa aacaagtttt
1441 tctgtcaaga agatgatcag accttggatc agatgaactc ttagaaatga aggcagaaaa
1501 atgtcattga gtaatatagt gactatgaac ttctctcaga cttactttac tcattttttt
1561 aatttattat tgaaattgta catatttgtg gaataatgta aaatgttgaa taaaaatatg
1621 tacaagtgtt gttttttaag ttgcactgat attttacctc ttattgcaaa atagcatttg
1681 tttaagggtg atagtcaaat tatgtattgg tggggctggg taccaatgct gcaggtcaac
1741 agctatgctg gtaggctcct gcctgtgtgg aaccactgac tactggctct cattgacttc
1801 cttactaagc atagcaaaca gaggaagaat ttgttatcag taagaaaaag aagaactata
1861 tgtgaatcct cttctttaca ctgtaattta gttattgatg tataaagcaa ctgttatgaa
1921 ataaagaaat tgcaataact ggcaaaaaaa aaaaaaaaaa aaaaaaaa
```

TABLE I
Histopathological data of Fus1^{+/-} and Fus1−/− mice that died between 3 to 14
months.

| Genotype | Number | Age/Gender^{a} | Histopathological abnormalities |
|---|---|---|---|
| Fus1^{+/-} | 3 | 7/F; 8/F; 12/F | Follicular cell lymphoma 2 4.5/F; 5.5/M |
| | | | Thymus atrophy, spleen depletion, bone marrow |
| | | | congestion |
| | 1 | 3.5/M | Thymus atrophy, spleen depletion, bone |
| | | | marrow congestion, kidney-tubular cell |
| | | | apoptosis and necrosis, heart-atrial pericardis, |
| | | | ventricular endocardis |
| | 1 | 3.5/M | Colorectal prolapse, spleen-marked |
| | | | erythropoiesis |
| | 2 | 12/F; 14F | Severe anemia, fibrinoid arteritis in multiple |
| | | | tissues, kidney glomerulonephritis (++), |
| | | | nephropathy (++), severe anemia |
| | 1 | 6/M | Head abscess, systemic infection |
| | 1 | 10/F | Severe anemia |
| Fus1^{-/-} | 2 | 5/M; 10/M | Thymus atrophy, spleen depletion, bone |
| | | | marrow congestion |
| | 1 | 10/M | Colorectal prolapse, hyperplasia in rectum and thymus |
| | 4 | 6.5/M; 9/F; | Kidney glomerulonephritis (+++), |
| | | 13/M | nephropathy (+++), tubular cast (+++), heart |
| | | | hemorrhage in aorta, arteritis |
| | 1 | 7/F | Follicular and histicioid lymphoma, arteritis in |
| | | | multiple tissues, kidney glomerulonephritis |
| | | | (+++), heart cardiomyopathy |
| | 1 | 6/M | Foci of inflammation in lung and liver |
| | 1 | 13/F | Invasive skin-squamus cell carcinoma, liver |
| | | | hepatocellular adenoma |

^{a}Age is indicated in months

TABLE II
Tissue localization of hemangioma/hemaniosarcomas in old mice

| | Num- | | | Heman- |
|---|---|---|---|---|
| BOS2 _498211.1Genotype | ber | Gender | Hemangioma | giosarcoma |
| WT | 397 | M | | Liver |
| Fus1^{+/-} | 123 | M | | Liver, spleen |
| | 149 | M | | Testis |
| | 376 | M | | Spleen |
| | 9 | F | Thymus | |
| | 46 | F | | Liver |
| | 82 | F | Liver | |
| | 85 | F | Uterus | |
| | 305 | F | | Ovary |
| | 210 | F | Spleen | |
| Fus1^{-/-} | 378 | M | | Spleen |
| | 92 | F | Bone | |
| | | | marrow | |
| | 112 | F | Liver | Liver, uterus |
| | 170 | F | Liver | |
| | 205 | F | | Uterus, bone |
| | | | | marrow |
| | 286 | F | | Liver, |
| | | | | spleen, |
| | | | | stomach |
| | 394 | F | | Liver Spleen |

TABLE III
Incidence of spontaneous tumors and severe abnormalities in Fus1^{+/-} and
Fus1^{-/-} aged mice (up to 2 years of age)

| | | Incidence of the pathological parameters considered in this study^{a} | | | | |
|---|---|---|---|---|---|---|
| | | | Glomerulonephritis | | | |
| | | | (++) and/or | Hemangioma/ | | Other |
| Genotype | Gender | Vasculitis | nephropathies | Hemangiosarcoma | Lymphoma | malignancies |
| WT | M | 1/15 (7) | 0/15 (0) | 1/15 (7) | 3/15 (20) | 2/15 (13) |
| | F | 0/12 (0) | 1/12 (8) | 0/12 (0) | 3/12 (25) | 4/12 (30) |
| | Total | 1/27 (4) | 1/27 (4) | 1/27 (4) | 6/27 (22) | 6/27 (22) |
| Fus1^{+/-} | M | 6/18 (33) | 6/18 (33) | 3/18 (17) | 2/18 (11) | 3/18 (17) |
| | F | 4/21 (19) | 4/21 (19) | 6/21 (29) | 9/21 (43) | 10/21 (48) |
| | Total | 10/39 (26) | 10/39 (26) | 9/39 (23) | 11/39 (28) | 13/39 (33) |
| Fus1^{-/-} | F | 2/11 (18) | 2/11 (18) | 1/11 (9) | 4/11 (36) | 0/11 (0) |
| | M | 4/14 (29) | 3/14 (23) | 7/14 (46) | 5/14 (36) | 6/14 (42) |
| | Total | 6/25 (24) | 5/25 (20) | 8/25 (32) | 9/25 (36) | 6/25 (24) |

^{a}Data indicate the number of mice presenting one of the pathological parameters considered, compared to the total number of mice investigated for this one. The corresponding percentages are mentioned ( ).

TABLE IV
The NK cell compartment of Fus1^{-/-} mice

| | | | NK cells (%)^{a,b} | | | | |
|---|---|---|---|---|---|---|---|
| | Lymphocytes (×10⁶)^{b} | | Total | | CD94⁺ | | Ly49G⁺ |
| | WT | Fus1^{-/-} | WT | Fus1^{-/-} | WT | Fus1^{-/-} | WT |
| No stimulation | | | | | | | |
| BM | 12.8 ± 1.51 | 8.60 ± 0.89 | 4.2 ± 0.5 | 3.5 ± 0.5 | 75 ± 3 | 61 ± 9 | 35 ± 6 |
| Liver | 2.70 ± 0.30 | 1.24 ± 0.38 | 6.0 ± 0.7 | 6.4 ± 0.5 | 77 ± 4 | 71 ± 6 | 38 ± 3 |
| Spleen | 45.6 ± 3.07 | 22.6 ± 5.63 | 3.1 ± 0.3 | 2.6 ± 0.3 | 73 ± 0 | 64 ± 8 | 43 ± 2 |
| IL-15 | | | | | | | |
| stimulation^{c} | | | | | | | |
| BM | 11.3 ± 1.40 | 11.9 ± 1.30 | 3.6 ± 0.4 | 3.2 ± 0.3 | 78 ± 3 | 63 ± 4 | 49 ± 1 |
| Liver | 2.18 ± 0.36 | 1.86 ± 0.56 | 4.3 ± 0.9 | 9.3 ± 1.2 | 83 ± 3 | 80 ± 1 | 37 ± 4 |
| Spleen | 35.1 ± 2.17 | 39.8 ± 2.70 | 3.0 ± 0.3 | 5.9 ± 0.4 | 74 ± 4 | 68 ± 1 | 45 ± 1 |

^{a}NK cells are defined as CD3⁻DX5⁺ lymphocytes.
^{b}Results are expressed as mean ± SEM from at least five experiments.
^{c}The flow cytometry analysis four days after injecting 5 μg of pCMV-SPORT6 as a source of mouse IL-15 cDN

### CITED DOCUMENTS

The following documents are referred to above by reference to the below sequential numbering with such number designations above generally set forth within parentheses (i.e. ( ).
1. Lerman, M. I., and J. D. Minna. 2000. The 630-kb lung cancer homozygous deletion region on human chromosome 3p21.3: identification and evaluation of the resident candidate tumor suppressor genes. The International Lung Cancer Chromosome 3p21.3 Tumor Suppressor Gene Consortium. Cancer Res. 60:6116-6133.
2. Kondo, M., L. Ji, C. Kamibayashi, Y. Tomizawa, D. Randle, Y. Sekido, J. Yokota, V. Kashuba, E. Zabarovsky, I. Kuzmin, M. Lerman, J. Roth, and J. D. Minna. 2001. Overexpression of candidate tumor suppressor gene FUS1 isolated from the 3p21.3 homozygous deletion region leads to G1 arrest and growth inhibition of lung cancer cells. Oncogene 20:6258-6262.
3. Ito, I., L. Ji, F. Tanaka, Y. Saito, B. Gopalan, C. D. Branch, K. Xu, E. N. Atkinson, B. N. Bekele, L. C. Stephens, J. D. Minna, J. A. Roth, and R. Ramesh. 2004. Liposomal vector mediated delivery of the 3p FUS1 gene demonstrates potent antitumor activity against human lung cancer in vivo. Cancer Gene Ther. 11:733-739.
4. Hanahan, D., R. A. Weinberg. 2000. The hallmarks of cancer. Cell 100:57 70.
5. Dunn, G. P., L. J. Old, and R. D. Schreiber. 2004. The three Es of cancer immunoediting. Annu. Rev. Immunol. 22:329-360. Review.
6. Dunn, G. P., L. J. Old, and R. D. Schreiber. 2004. The immunobiology of cancer immunosurveillance and immunoediting. Immunity. 21:137-148. Review.
7. Yokoyama, W. M., S. Kim, and A. R. French. 2004. The dynamic life of natural killer cells. Annu. Rev. Immunol. 22:405-429.
8. Smyth, M. J., N. Y. Crowe, and D. I. Godfrey. 2001. NK cells and NKT cells collaborate in host protection from methylcholanthrene-induced fibrosarcoma. Int. Immunol. 13:459-463.
9. Vosshenrich, C. A., T. Ranson, S. I. Samson, E. Corcuff, F. Colucci, E. E. Rosmaraki, and J. P. Di Santo. 2005. Roles for Common Cytokine Receptor {gamma}-Chain-Dependent Cytokines in the Generation, Differentiation, and Maturation of NK Cell Precursors and Peripheral NK Cells in Vivo. J. Immunol. 174: 1213-1221.
10. Lodolce, J. P., D. L. Boone, S. Chai, R. E. Swain, D. Themistocles, S. Trettin, and A. Ma. 1998. IL-15 receptor maintains lymphoid homeostasis by supporting lymphocyte homing and proliferation. Immunity 9:669-676.
11. Kennedy, M. K., M. Glaccum, S. N. Brown, E. A. Butz, J. L. Viney, M. Embers, N. Matsuki, K. Charrier, L. Sedger, C. R. Willis, K. Brasel, P. J. Morrissey, K. Stocking, J. C. Schuh, S. Joyce, and J. J. Peschon. 2000. Reversible defects in natural killer and memory CD8 T cell lineages in interleukin 15-deficient mice. J. Exp. Med. 191:771-780.
12. Uno F., J. Sasaki, M. Nishizaki, G. Carboni, K. Xu, E. N. Atkinson, M. Kondo, J. D. Minna, J. A. Roth, and L. Ji. 2004. Myristoylation of the Fus1 protein is required for tumor suppression in human lung cancer cells. Cancer Res. 64: 2969-2976.
13. Haines, D. C., S. Chattopadhyay, J. M. Ward. 2001. Pathology of aging B6; 129 mice. Toxicol. Pathol. 29:653-661.
14. Ramdial, P. K., and R. Chetty. 1999. Vasculitis-induced membranous fat necrosis. J. Cutan. Pathol. 26:405-410.
15. Ng, P. P., S. H. Tan, and T. Tan. 2002. Lupus erythematosus panniculitis: a clinicopathologic study. Int. J. Dermatol. 41: 488-490.
16. Sherer, Y., A. Gorstein, M. J. Fritzler, and Y. Shoenfeld. 2004. Autoantibody explosion in systemic lupus erythematosus: more than 100 different antibodies found in SLE patients. Semin. Arthritis Rheum. 34:501-537. Review.
17. Seo, P., and J. H. Stone. 2004. The antineutrophil cytoplasmic antibody-associated vasculitides. Am. J. Med. 117:39-50. Review.
18. Bjornsson, J. 2002. Histopathology of primary vasculitic disorders. In: Inflammatory diseases of blood vessels. Hoffman G S, Weyand C M, editors. New York: Marcel Dekker 255-265.
19. Dorfman, J. R., and R. H. Raulet. 1998. Acquisition of Ly49 receptor expression by developing natural killer cells. J. Exp. Med. 187: 609-618.
20. Williams, N. S., A. Kubota, M. Bennett, V. Kumar, and F. Takei. 2000. Clonal analysis of NK cell development from bone marrow progenitors in vitro: orderly acquisition of receptor gene expression. Eur. J. Immunol. 30:2074-2082.
21. Kim, S., K. Jizuka, H. S. Kang, A. Dokun, A. R. French, S. Greco, and M. V. Yokoyama. 2002. In vivo developmental stages in murine natural killer cell maturation. Nat. Immunol. 3: 523-528.
22. Makrigiannis, A. P., and S. K. Anderson. 2003. Regulation of natural killer cell function. Cancer Biol. Ther. 2:610-616. Review.
23. Colucci, F., M. A. Caligiuri, and J. P. Di Santo. 2003. What does it take to make a killer? Nat. Rev. Immunol. 3:413-425.
24. Sivakumar, P. V., N. S. Williams, I. J. Puzanov, J. D. Schatzle, M. Bennett, and V. Kumar. 1998. Development of self-recognition systems in natural killer cells. Adv. Exp. Med. Biol. 452:1-12.
25. Perera, L. P. 2000. Interleukin 15: its role in inflammation and immunity. Arch. Immunol. Ther. Exp. (Warsz).48:457-464. Review.
26. Ohteki, T., K. Suzue, C. Maid, T. Ota, and S. Koyasu. 2001. Critical role of IL-15-IL-15R for antigen-presenting cell functions in the innate immune response. Nat. Immunol. 2:1138-1143.
27. Degli-Esposti, M. A, and M. J. Smyth. 2005. Close encounters of different kinds: dendritic cells and NK cells take centre stage. Nat. Rev. Immunol. 5:112-124.
28. Williams, N. S., J. Klem, I. J. Puzanov, P. V. Sivakumar, J. D. Schatzle, M. Bennett, and V. Kumar. 1998. Natural killer cell differentiation: insights from knockout and transgenic mouse models and in vitro systems. Immunol. Rev. 165:47-61.
29. Fehniger, T. A., and M. A. Caligiuri. 2001. Interleukin 15: biology and relevance to human disease. Blood. 97:14-22.
30. Liu, C. C., B. Perussia, and J. D. Young. 2000. The emerging role of IL-15 in NK-cell development. Immunol. Today. 21:113-116. Review.
31. Schluns, K. S., Nowak, E. C., Cabrera-Hernandez, A., Puddington, L., Lefrancois, L., and Aguila, H. L. 2004. Distinct cell types control lymphoid subset development by means of IL-15 and IL-15 receptor alpha expression. Proc. Natl. Acad. Sci. U S A. 101:5616-5621.
32. Ogasawara, K., S. Hida, N. Azimi, Y. Tagaya, T. Sato, T. Yokochi-Fukada, T. A. Waldman, T. Taniguchi, and S. Taki. 1998. Requirement for IRF-1 in the microenvironment supporting development of natural killer cells. Nature 391:700-703.
33. Ohteki, T., H. Yoshida, T. Matsuyama, G. S. Duncan, T. W. Mak, P. S. Ohashi. 1998. The transcription factor interferon regulatory factor 1 (IRF-1) is important during the maturation of natural killer 1.1⁺ T cell receptor-/β₁ (NK1⁺ T) cells, natural killer cells, and intestinal intraepithelial T cells. J. Exp. Med. 187:967-972.
34. Wilder, J. A., C. Y. Koh, and D. Yuan. 1996. The role of NK cells during in vivo antigen-specific antibody responses. J. Immunol. 156:146-152.
35. Kim, S., K. Iizuka, H. L. Aguila, I. L. Weissman, and Yokoyama W. M. 2000. In vivo natural killer cell activities revealed by natural killer cell-deficient mice. Proc. Natl. Acad. Sci. U S A. 97:2731-2736.
36. Erkeller-Yusel, F., F. Hulstaart, I. Hannet, D. Isenberg, and P. Lydyard. 1993. Lymphocyte subsets in a large cohort of patients with systemic lupus erythematosus. Lupus. 2:227-231.
37. Baxter, A. G., and M. J. Smyth. 2002. The role of NK cells in autoimmune disease. Autoimmunity. 35:1-14. Review.
38. Kuper, H., H. O. Adami, and D. Trichopoulos. 2000. Infections as a major preventable cause of human cancer. J. Intern. Med. 248:171-183. Review.
39. Engle, S. J., J. B. Hoying, G. P. Boivin, I. Ormsby, P. S. Gartside, and T. Doetschman. 1999. Transforming growth factor beta1 suppresses nonmetastatic colon cancer at an early stage of tumorigenesis. Cancer Res. 59: 3379 3386.
40. Engle, S. J., I. Ormsby, S. Pawlowski, G. P. Boivin, J. Croft, E. Balish, and T. Doetschman. 2002. Elimination of colon cancer in germ-free transforming growth factor beta 1-deficient mice. Cancer Res. 62:6362 6366.
41. Varoczy, L., L. Gergely, M. Zeher, G. Szegedi, and A. Illes. 2002. Malignant lymphoma-associated autoimmune diseases—a descriptive epidemiological study. Rheumatol. Int. 22:233-237.
42. Jakobisiak, M., W. Lasek, and J. Golab. 2003. Natural mechanisms protecting against cancer. Immunol. Lett. 90: 103-122. Review.
43. Knudson, A. G. Jr. 1971. Mutation and cancer: statistical study of retinoblastoma. Proc. Natl. Acad. Sci. USA. 68:820-823.
44. Knudson, A. G. 1993. Anti-oncogenes and human cancer. Proc. Natl. Acad. Sci. USA. 90:10914-10921. Review.
45. Zhang, L., and Pagano, J. S. 2002. Structure and function of 1RF-7. J. Interferon. Cytokine Res. 22:95-101. Review.
46. Bonin, A., S. W. Reid, and Tessarollo, L. 2001. Isolation, microinjection, and transfer of mouse blastocysts. Methods Mol. Biol. 158:121-134.
47. Tessarollo, L. 2001. Manipulating mouse embryonic stem cells. Methods Mol. Biol. 158:47-63.
48. Ivanov, S. V., Ward, J. M., Tessarollo, L., McAreavey, D., Sachdev, V., Fananapazir, L., Banks, M. K., Morris, N., Djurickovic, D., Devor-Henneman, D. E., Wei, M. H., Alvord, G. W., Gao, B., Richardson, J. A., Minna, J. D., Rogawski, M. A., Lerman, M. I. 2004. Cerebellar ataxia, seizures, premature death, and cardiac abnormalities in mice with targeted disruption of the Cacna2d2 gene. Am. J. Pathol. 165:1007-1018.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

A number of embodiments of the invention have been described. Nevertheless, it will be understood that various modifications may be made without departing from the spirit and scope of the invention. Accordingly, other embodiments are within the scope of the following claims.

1-52. (canceled)

53. A method for altering cellular proliferation comprising contacting an immune cell with a FUS1 composition.

54. A method of treating, preventing, or alleviating a FUS1 immune related disorder in a subject, comprising:
administering a FUS1 composition to a subject suffering from or susceptible to a FUS1 immune related disorder.

55. The method of claim 54 wherein the FUS1 immune related disorder is an immune disorder associated with cancer.

56. The method of claim 54 wherein the FUS1 immune related disorder is one or more disorders selected from the group consisting of an autoimmune disease, anemia, inflammatory infiltrating of vessels, glomerulonephritis, circulating antibodies, vasculitis or NK maturation defect.

57. The method of claim 54 wherein the subject is identified as suffering from or susceptible to a FUS1 immune related disorder and the FUS1 composition is administered to the identified subject.

58. A method of predicting or diagnosing a FUS1 related immune disorder in a subject comprising:
determining the level of FUS1 expression in a sample from a subject and correlating the determined level to FUS1 related immune disorder status of the subject.

59. The method of claim 58 wherein the FUS1 immune related disorder is an immune disorder associated with cancer.

60. The method of claim 58 wherein the FUS1 immune related disorder is one or more disorders selected from the group consisting of an autoimmune disease, anemia, inflammatory infiltrating of vessels, glomerulonephritis, circulating antibodies, vasculitis or NK maturation defect.

58. The method of claim 58 wherein the subject is identified as suffering from or susceptible to a FUS1 immune related disorder and the FUS1 composition is administered to the identified subject.

59. A method of treating, preventing, or alleviating a FUS1 immune related disorder in a subject, comprising:
administering a therapeutically effective amount of an IL15 composition to a subject suffering from or susceptible to a FUS1 immune related disorder selected from anemia, inflammatory infiltrating of vessels, glomerulonephritis, circulating antibodies, vasculitis, NK maturation defect, or an immune disorder associated with cancer.

60. The method of claim 53 wherein the FUS1 composition comprises a substantially purified FUS1 polypeptide, or fragment or variant thereof.

61. The method of claim 53 wherein the FUS1 composition comprises a nucleic acid encoding a FUS1 polypeptide, or fragment or variant thereof.

62. The method of claim 60 wherein the FUS1 composition comprises a polypeptide that comprises one or more of SEQ ID NO. 1-13.

63. The method of claim 62 wherein the polypeptide further comprises a protein transduction domain.

64. The method of claim 63 wherein the protein transduction domain is one or more of the Drosophila homeotic transcription protein antennapedia (Antp), the herpes simplex virus structural protein VP22, or the human immunodeficiency virus 1 (HIV-1) transcriptional activator Tat protein.

65. A method for predicting or diagnosing a FUS1 immune related disorder in a subject comprising determining level of FUS1 expression in a sample from a subject.

66. The method of claim 65 wherein a reduced level of FUS1 in the sample indicates that subject has or is at risk of developing a FUS1 related disorder.

67. The method of claim 65 wherein reduced levels of IL15 in the sample indicates that the subject is at risk of developing or has a FUS1 related disorder.

68. The method of claim 65 further comprising obtaining a sample from the subject.

69. The method of claim 68 wherein the sample is one or more of a tissue sample, blood, sputum, bronchial washings, biopsy aspirate, or ductal lavage.

70. The method of claim 65 wherein determining comprises an immunoassay.

71. The method of claim 65 wherein the FUS1 immune related disorder is an autoimmune disease, anemia, a virus associated malignancy, inflammatory infiltrating of vessels, glomerulonephritis, circulating antibodies, vasculitis, or NK cell maturation defect.

72. The method of claim 53, wherein the immune system cell comprises one or more of an NK cell, T cell, B cell, activated T cell, activated B cell.


SEQUENCE LISTING (23 sequences)

SEQ ID NO: 1 - protein, 110 aa, Homo sapiens
MGASGSKARGLWPFASAAGGGGSEAAGAEQALVRPRGRAVPPFVFTRRGSMFYDEDGDLAHEFYEETIVTKNGQKRAKLRRVHKNLIPQGIVKLDHPRIHVDFPVILYEV

SEQ ID NO: 2 - protein, 110 aa, Mus sp.
MGASGSKARGLWPFASTPGGGGPEAAGSEQSLVRSRARAVPPFVFTRRGSMFYDEDGDLAHEFYEETIVTKNGQKRAKLRRVHKNLIPQGIVKLDPPRIHVDFPVILYEV

SEQ ID NO: 3 - protein, 110 aa, Rattus norvegicus
MGSSISKARGLCPFVSTTGVGSPVAEVAKQSLVRSRARAVPPFVFTRRGSMFYDEDGDLAHEFYEETIITKNGQKRSNLRRVRNNLIPQGIVKLERPQIHVDFPLILCEV

SEQ ID NO: 4 - protein, 111 aa, Danio rerio
MGGSGSKGKGYWPFSGSGGGDEPAKEGQEQSLSRVRSIRNATPFVFTRRSSLYFDEDGDLAHEFYEETVVTKNGRKKAKLKRIHKNLIPQGIIKLDHPCIHVDFPVVICEA

SEQ ID NO: 5 - protein, 106 aa, Xenopus laevis
MGGSASKARGLWPFSSTTSEAQPGNDDQSVTRMRKATPFIFTRRGSMYFDEDGDLAHEFYEETVVMKNGRKRAKLKRIQKNLRPQGIIRLDHPCLHVDFPVVICEV

SEQ ID NO: 6 - protein, 123 aa, Caenorhabditis elegans
MGLGSSKRKEEPPHKSEPKTVGRVKRAGARPDEMIAKYAEVLKTRGILPEYFLVHEAKSSQYIDEDGDVANEFYQETMSDGEKRRLCRLMKNLRPKGKERYAIPRLKHDIPVVIWEVQQPQET

SEQ ID NO: 7 - protein, 110 aa, Homo sapiens; identical to SEQ ID NO: 1

SEQ ID NO: 8 - protein, 110 aa, Artificial Sequence; identical to SEQ ID NO: 1
  Description of Artificial Sequence Synthetic construct

SEQ ID NO: 9 - protein, 96 aa, Artificial Sequence
  Description of Artificial Sequence Synthetic construct
LRCEQSGHGARHAPAALTWAPAGPKLGACGPSPRRPEAAAQRQQELSKLWCGLGAELCPPSYSRAAALCSMMRMGIWLTSSMRRQSSPRTGRSGPS

SEQ ID NO: 10 - protein, 81 aa, Artificial Sequence [sequence omitted]

SEQ ID NO: 11 - protein, 80 aa, Artificial Sequence
  Description of Artificial Sequence Synthetic construct
LNKSRMRTGLGGCLDGVLCSMFYDEDGDLAHEFYEETIVTKNGQKRAKLRRVHKNLIPQGIVKLDHPRIHVDFPVILYEV

SEQ ID NO: 12 - protein, 110 aa, Homo sapiens; identical to SEQ ID NO: 1

SEQ ID NO: 13 - protein, 110 aa, Homo sapiens; identical to SEQ ID NO: 1

SEQ ID NO: 14 - DNA, 1691 bp, Homo sapiens
tgcggccgcgtttccgtggagacagccgagcctgcggaaggcggcggcggcggcacctgcgatcagcggctggggcaggttatggtagtgcggactgcggtgtgagcagagcggccacggggcccgccatgcgccggcggccctgacatgggcgccagcgggtccaaagctcggggcctgtggcccttcgcctcggcggccggaggcggcggctcagaggcagcaggagctgagcaagctttggtgcggcctcggggccgagctgtgccccccttcgtattcacgcgccgcggctctatgttctatgatgaggatggggatctggctcacgagttctatgaggagacaatcgtcaccaagaacgggcagaagcgggccaagctgaggcgagtgcataagaatctgattcctcagggcatcgtgaagctggatcacccccgcatccacgtggatttccctgtgatcctctatgaggtgtgaccctgggaggtggcagacagaagcaccccctgccccggcaagaaactcccaggctcaatcaaggtgtggcttccattgaggagcccaggctggggccacaaccctgaataaactctgttggcccataaccttcagctgtgagcgggtcggtcccacagtattggttgggtgttggtttgtgtgtggacaagaggtggttggtgggtggtgaaggctaatggcagagttagcaccccactctcccaagccacccctgcaagcagcatagcagggcatataccagtcaggaatgcccgttacctggttccttgcctggtctgctttcttccaagtttgcctggggcctagccctgctagaggctacagcactttacaagcaaggtatgctttcttccagcccctaggctgtgggcactgtatacaagtaggaacttcctttccttcacttcccttttaacccctagtcagagcatttcagccgtttgctacctcgattcctcctgtgttggacagaggctgggggcagtgccagcctgattcttccgacctacctgccatttgttcccgccttcagatggatggacagtttgctggctattgataggagtggggactgggtgggggcttctccctctacccagggctgggctgatccccctactgcaactaactgttgccccccaaccccgaacccccagttgaggagttgagagagtgcaggctggggtcaggacaggctgcggatgcttgtgcctatggggagttactccaacccacctattctgtctaatctccatggctttgcaccaaatcctccacccctccaattgggaggggactgttcaccaccttgtggtaagggacaacaccctaaggctggtgccagtagttatgagtagcctaccaccccctcccttacagtaacccccaccccttcaggatcagtcaagggaaagcactagaacccctgggtagggaaagaaaggagggaaaaaccataaaaggaatacttataatgtgaaggtttgtaaatagtccatgatgatgtcgtggcagagtctgatttctatatagaggtgactttttttttaagtactgtgcaagctctgtgcttctataatgtgggaaatggcttggggaggatggcccctagcttaggaagactgttgtgttatttgttcaatttcaataaaatgatttgtagatcctgc

SEQ ID NO: 15 - DNA, 1666 bp, Homo sapiens
ggcacgaggctgcgatcagcggctggggcaggttatggtagtgcggactgcggtgtgagcagagcggccacggggcccgccatgcgccggcggccctgacatgggcgccagcgggtccaaagctcggggcctgtggcccttcgcctcggcggccggaggcggcggctcagaggcagcaggagctgagcaagctttggtgcggcctcggggccgagctgtgccccccttcgtattcacgcgccgcggctctatgttctatgatgaggatggggatctggctcacgagttctatgaggagacaatcgtcaccaagaacgggcagaagcgggccaagctgaggcgagtgcataagaatctgattcctcagggcatcgtgaagctggatcacccccgcatccacgtggatttccctgtgatcctctatgaggtgtgaccctgggaggtggcagacagaagcaccccctgccccggcaagaaactcccaggctcaatcaaggtgtggcttccattgaggagcccaggctggggccacaaccctgaataaactctgttggcccataaccttcagctgtgagcgggtcggtcccacagtattggttgggtgttggtttgtgtgtggacaagaggtggttggtgggtggtgaaggctaatggcagagttagcaccccactctcccaagccacccctgcaagcagcacagcagggcatataccagtcaggaatgcccgttacctggttccttgcctggtctgctttcttccaagtttgcctggggcctagccctgctagaggctacagcactttacaagcaaggtatgctttcttccagcccctaggctgtgggcactgtatacaagtaggaacttcctttccttcacttcccttttaacccctagtcagagcatttcagccgtttgctacctcgattcctcctgtgttggacagaggctgggggcagtgccagcctgattcttccgacctacctgccatttgttcccgccttcagatggatggacagtttgctggctattgataggagtggggactgggtgggggcttctccctctacccagggctgggctgatccccctactgcaactaactgttgccccccaaccccgaacccccagttgaggagttgagagagtgcaggctggggtcaggacaggctgcggatgcttgtgcctatggggagttactccaacccacctattctgtctaatctccatggctttgcaccaaatcctccacccctccaattgggaggggactgttcaccaccttgtggtaagggacaacaccctaaggctggtgccagtagttatgagtagcctaccaccccctcccttacagtaacccccaccccttcaggatcagtcaagggaaagcactagaacccctgggtagggaaagaaaggagggaaaaaccataaaaggaatacttataatgtgaaggtttgtaaatagtccatgatgatgtcgtggcagagtctgatttctatatagaggtgactttttttttaagtactgtgcaagctctgtgcttctataatgtgggaaatggcttggggaggatggcccctagcttaggaagactgttgtgttatttgttcaatttcaataaaatgatttgtagatcctgcaaaaaaaaaaaaaaaaaaaaaa

SEQ ID NO: 16 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - DNA, 1968 bp, Homo sapiens
actccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggaggtacagagctcggcttctttgccttgggaggggagtggtggtggttgaaagggcgatggaattttccccgaaagcctacgcccagggcccctcccagctccagcgttaccctccggtctatcctactggccgagctgccccgccttctcatggggaaaacttagccgcaacttcaatttttggtttttcctttaatgacacttctgaggctctcctagccatcctcccgcttccggaggagcgcagatcgcaggtccctttgcccctggcgtgcgactccctactgcgctgcgctcttacggcgttccaggctgctggctagcgcaaggcgggccgggcaccccgcgctccgctgggagggtgagggacgcgcgtctggcggccccagccaagctgcgggtttctgagaagacgctgtcccgcagccctgagggctgagttctgcacccagtcaagctcaggaaggccaagaaaagaatccattccaatatatggccatgtggctctttggagcaatgttccatcatgttccatgctgctgacgtcacatggagcacagaaatcaatgttagcagatagccagcccatacaagatcgtattgtattgtaggaggcatcgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgcctgtgtggaaccactgactactggctctcattgacttccttactaagcatagcaaacagaggaagaatttgttatcagtaagaaaaagaagaactatatgtgaatcctcttctttacactgtaatttagttattgatgtataaagcaactgttatgaaataaagaaattgcaataactggcaaaaaaaaaaaaaaaaaaaaaaaaa

SEQ ID NO: 18 - DNA, 40 bp, Artificial Sequence
  Description of Artificial Sequence Synthetic oligonucleotide
nnnncctacggccgcaagaaacgccgccagcgccgccgca

SEQ ID NO: 19 - DNA, 39 bp, Artificial Sequence
  Description of Artificial Sequence Synthetic oligonucleotide
nnnngcggcggcgctggcggcgtttcttgcggccgtagg

SEQ ID NO: 20 - protein, 110 aa, Homo sapiens; identical to SEQ ID NO: 1

SEQ ID NO: 21 - protein, 120 aa, Artificial Sequence
  Description of Artificial Sequence Synthetic construct
YGRKKRRQRRRMGASGSKARGLWPFASAAGGGGSEAAGAEQALVRPRGRAVPPFVFRRGSMFYDEDGDLAHEFYEETIVTKNGQKRAKLRRVHKNLIPQGIVKLDHPRIHVDFPVILYEV

SEQ ID NO: 22 - protein, 121 aa, Artificial Sequence
  Description of Artificial Sequence Synthetic construct
MGASGSKARGLWPFASAAGGGGSEAAGAEQALVRPRGRAVPPFVFTRRGSMFYDEDGDLAHEFYEETIVTKNGQKRAKLRRVHKNLIPQGIVKLDHPRIHVDFPVILYEVRRRQRRKKRGY

SEQ ID NO: 23 - protein, 11 aa, Human immunodeficiency virus
YGRKKRRQRRR